SEQUENCE LISTING (1 sequence)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

(19) United States
(12) Patent Application Publication
(10) Pub. No.: US 2026/0287581 A1
(43) Pub. Date: Sep. 24, 2026

(54) METHOD AND DEVICE FOR ESTIMATING AMOUNT OF TAU PROTEIN ACCUMULATION IN SUBJECT'S BRAIN

(71) Applicant: National Institutes for Quantum Science and Technology, Chiba (JP)

(72) Inventors: Takahiko TOKUDA, Chiba (JP); Harutsugu TATEBE, Chiba (JP)

(21) Appl. No.: 19/474,541

(22) PCT Filed: Apr. 2, 2024
(86) PCT No.: PCT/JP2024/013555

§ 371 Date: Oct. 10, 2025

(30) Foreign Application Priority Data
Apr. 13, 2023 (JP) 2023-065877
Dec. 25, 2023 (JP) 2023-218481

(51) Int. Cl. G01N 33/543; G01N 33/541; G01N 33/68

(52) U.S. Cl. CPC G01N 33/54313; G01N 33/541; G01N 33/6896; G01N 2333/4709; G01N 2470/06; G01N 2800/2821

(57) ABSTRACT

The present disclosure relates to a method for estimating the amount of a tau protein accumulated in the brain. One of a capture antibody and a detection antibody which are used in the method is an antibody which recognizes an amino acid sequence in a middle part of a phosphorylated tau protein, and the other is an antibody which recognizes a phosphorylated epitope including a phosphorylated amino acid residue of the phosphorylated tau protein.

### TECHNICAL FIELD

The present invention relates to a method, a device, etc. for estimating the amount of a tau protein accumulated in the brain of a subject by detecting a phosphorylated tau protein in a biological sample collected from the subject.

### BACKGROUND ART

A phosphorylated tau protein is considered to be one of candidate biomarkers for tauopathies, such as Alzheimer's disease. To date, various research and development efforts have been undertaken regarding the use of a phosphorylated tau protein as a biomarker.

For example, Patent Literature 1 and Non-Patent Literature 1 each disclose a method for measuring a phosphorylated tau protein with the use of Simoa (trademark), which is known as an ultra-sensitive digital ELISA device. Non-Patent Literature 2 discloses that a phosphorylated tau protein in which a specific amino acid is phosphorylated is useful as a biomarker for Alzheimer's disease.

Non-Patent Literature 3 reports that the amount of a phosphorylated tau protein which is measured by previously reported methods for measuring a phosphorylated tau protein reflects the amount of amyloid accumulated in the brain, rather than the amount of a tau protein accumulated in the brain.

### CITATION LIST

### Patent Literature

[Patent Literature 1]
   Japanese Patent Application Publication Tokukai No. 2019-27952

### Non-Patent Literature

[Non-patent Literature 1]
   Tatebe H, et al. Quantification of plasma phosphorylated tau to use as a biomarker for brain Alzheimer pathology: pilot case-control studies including patients with Alzheimer's disease and down syndrome. Mol Neurodegener. 2017; 12(1):63
[Non-patent Literature 2]
   Karikari T K, et al. Blood phosphorylated tau 181 as a biomarker for Alzheimer's disease: a diagnostic performance and prediction modelling study using data from four prospective cohorts. Lancet Neurol. 2020; 19(5):422-433
[Non-patent Literature 3]
   Therriault J, et al. Association of Phosphorylated Tau Biomarkers With Amyloid Positron Emission Tomography vs Tau Positron Emission Tomography. JAMA Neurol. 2022 Dec. 12

### SUMMARY OF INVENTION

### Technical Problem

Before the onset of mild cognitive impairment, the accumulation of amyloid in the brain is observed. As cognitive decline progresses, the accumulation of an abnormal tau protein, such as a phosphorylated tau protein, in the brain is observed. Since the accumulation of amyloid in the brain is already saturated by the time of the onset of mild cognitive impairment, it is necessary to measure the amount of the abnormal tau protein accumulated in the brain, in order to prevent the onset of or treat cognitive impairment, such as mild cognitive impairment, or neurodegenerative disorders.

It is known that the amount of an abnormal tau protein accumulated in the brain can be measured by tau positron emission tomography (PET). However, measurement by tau PET is expensive, and facilities in which tau PET can be used are very limited. Additionally, measurement by tau PET raises concerns regarding invasiveness due to radiation.

Accordingly, there is a demand for the development of a method etc. that make it possible to estimate, in a simple manner without the use of tau PET, the amount of an abnormal tau protein accumulated in the brain.

The object of an aspect of the present invention is to realize a method, a device, etc. each of which makes it possible to predict, in a simple manner, the amount of an abnormal tau protein accumulated in the brain.

### Solution to Problem

In order to attain the above object, the present invention includes the following aspects:
A method for estimating an amount of a tau protein accumulated in a brain of a subject, the method including: a preparing step of forming, on a capture bead, an immune complex of a phosphorylated tau protein in a biological sample collected from a subject, a capture antibody, and a detection antibody in the presence of a non-capture bead to prepare a measurement sample containing the non-capture bead and the capture bead to which the immune complex binds;
   a detecting step of detecting a signal derived from the immune complex in the measurement sample; and
   an estimating step of estimating, based on intensity of the signal, an amount of a tau protein accumulated in a brain of the subject which amount is quantified by tau PET, wherein
   the non-capture bead does not bind to the immune complex,
   one of the detection antibody and the capture antibody is an antibody which recognizes an amino acid sequence from position 151 to position 244 in an amino acid sequence represented by SEQ ID NO. 1,
   the other of the detection antibody and the capture antibody is an antibody which recognizes a phosphorylated epitope including a phosphorylated amino acid residue in the amino acid sequence represented by SEQ ID NO. 1, and
   the intensity of the signal does not correlate with an amount of amyloid accumulated in the brain of the subject which amount is quantified by amyloid PET.
A device which estimates an amount of a tau protein accumulated in a brain of a subject, the device including a processing section, wherein
   regarding a measurement sample which has been prepared by forming, on a capture bead, an immune complex of a phosphorylated tau protein in a biological sample collected from a subject, a detection antibody, and a capture antibody in the presence of a non-capture bead and which contains the non-capture bead and the capture bead to which the immune complex binds, the processing section obtains a signal derived from the immune complex,
   the processing section estimates, based on intensity of the signal, an amount of a tau protein accumulated in a brain of the subject which amount is quantified by tau PET,
   the non-capture bead does not bind to the immune complex,
   one of the detection antibody and the capture antibody is an antibody which recognizes an amino acid sequence from position 151 to position 244 in an amino acid sequence represented by SEQ ID NO. 1,
   the other of the detection antibody and the capture antibody is an antibody which recognizes a phosphorylated epitope including a phosphorylated amino acid residue in the amino acid sequence represented by SEQ ID NO. 1, and
   the intensity of the signal does not correlate with an amount of amyloid accumulated in the brain of the subject which amount is quantified by amyloid PET.
A device which obtains information on whether or not a subject is affected by a tauopathy, based on intensity of a signal derived from a phosphorylated tau protein in a biological sample collected from the subject,
   the device including a processing section, wherein
   regarding a measurement sample which has been prepared by forming, on a capture bead, an immune complex of a phosphorylated tau protein in a biological sample collected from a subject, a detection antibody, and a capture antibody in the presence of a non-capture bead and which contains the non-capture bead and the capture bead to which the immune complex binds, the processing section obtains a signal derived from the immune complex,
   the processing section estimates, based on intensity of the signal, an amount of a tau protein accumulated in a brain of the subject which amount is quantified by tau PET,
   the processing section obtains information for evaluating a degree of progression of a tauopathy in the subject, based on the amount of the tau protein accumulated in the brain of the subject which amount has been estimated,
   the non-capture bead does not bind to the immune complex,
   one of the detection antibody and the capture antibody is an antibody which recognizes an amino acid sequence from position 151 to position 244 in an amino acid sequence represented by SEQ ID NO. 1,
   the other of the detection antibody and the capture antibody is an antibody which recognizes a phosphorylated epitope including a phosphorylated amino acid residue in the amino acid sequence represented by SEQ ID NO. 1, and
   the intensity of the signal does not correlate with an amount of amyloid accumulated in the brain of the subject which amount is quantified by amyloid PET.
A device which obtains information on whether or not a subject is at Braak stage I/II or Braak stage III or higher, based on intensity of a signal derived from a phosphorylated tau protein in a biological sample collected from the subject,
   the device including a processing section, wherein
   regarding a measurement sample which has been prepared by forming, on a capture bead, an immune complex of a phosphorylated tau protein in a biological sample collected from a subject, a detection antibody, and a capture antibody in the presence of a non-capture bead and which contains the non-capture bead and the capture bead to which the immune complex binds, the processing section obtains a signal derived from the immune complex,
   the processing section estimates, based on intensity of the signal, an amount of a tau protein accumulated in a brain of the subject which amount is quantified by tau PET,
   the processing section obtains information on whether or not the subject is at Braak stage I/II or Braak stage III or higher, based on the amount of the tau protein accumulated in the brain of the subject which amount has been estimated,
   the non-capture bead does not bind to the immune complex,
   one of the detection antibody and the capture antibody is an antibody which recognizes an amino acid sequence from position 151 to position 244 in an amino acid sequence represented by SEQ ID NO. 1,
   the other of the detection antibody and the capture antibody is an antibody which recognizes a phosphorylated epitope including a phosphorylated amino acid residue in the amino acid sequence represented by SEQ ID NO. 1, and
   the intensity of the signal does not correlate with an amount of amyloid accumulated in the brain of the subject which amount is quantified by amyloid PET.
A computer program for estimating an amount of a tau protein accumulated in a brain of a subject, the computer program causing a computer to execute:
   a step of, regarding a measurement sample which has been prepared by forming, on a capture bead, an immune complex of a phosphorylated tau protein in a biological sample, a detection antibody, and a capture antibody in the presence of a non-capture bead and which contains the non-capture bead and the capture bead to which the immune complex binds, obtaining a signal derived from the immune complex, wherein
   the biological sample is collected from a subject,
   the non-capture bead does not bind to the immune complex,
   one of the detection antibody and the capture antibody is an antibody which recognizes an amino acid sequence from position 151 to position 244 in an amino acid sequence represented by SEQ ID NO. 1, and
   the other of the detection antibody and the capture antibody is an antibody which recognizes a phosphorylated epitope including a phosphorylated amino acid residue in the amino acid sequence represented by SEQ ID NO. 1; and
   a step of estimating, based on intensity of the signal, an amount of a tau protein accumulated in a brain of the subject which amount is quantified by tau PET, wherein
   the intensity of the signal does not correlate with an amount of amyloid accumulated in the brain of the subject which amount is quantified by amyloid PET.
A computer program for obtaining information for evaluating a degree of progression of a tauopathy, the computer program causing a computer to execute:
   a step of, regarding a measurement sample which has been prepared by forming, on a capture bead, an immune complex of a phosphorylated tau protein in a biological sample, a detection antibody, and a capture antibody in the presence of a non-capture bead and which contains the non-capture bead and the capture bead to which the immune complex binds, obtaining a signal derived from the immune complex, wherein
   the biological sample is collected from a subject,
   the non-capture bead does not bind to the immune complex,
   one of the detection antibody and the capture antibody is an antibody which recognizes an amino acid sequence from position 151 to position 244 in an amino acid sequence represented by SEQ ID NO. 1, and
   the other of the detection antibody and the capture antibody is an antibody which recognizes a phosphorylated epitope including a phosphorylated amino acid residue in the amino acid sequence represented by SEQ ID NO. 1;
   a step of estimating, based on intensity of the signal, an amount of a tau protein accumulated in a brain of the subject which amount is quantified by tau PET; and
   a step of obtaining information for evaluating a degree of progression of a tauopathy in the subject, from whom/which the biological sample has been collected, based on the amount of the tau protein accumulated in the brain of the subject which amount has been estimated, wherein
   the intensity of the signal does not correlate with an amount of amyloid accumulated in the brain of the subject which amount is quantified by amyloid PET.
A computer program for obtaining information on whether or not a subject is at Braak stage I/II or Braak stage III or higher, the computer program causing a computer to execute:
   a step of, regarding a measurement sample which has been prepared by forming, on a capture bead, an immune complex of a phosphorylated tau protein in a biological sample, a detection antibody, and a capture antibody in the presence of a non-capture bead and which contains the non-capture bead and the capture bead to which the immune complex binds, obtaining a signal derived from the immune complex, wherein
   the biological sample is collected from a subject,
   the non-capture bead does not bind to the immune complex,
   one of the detection antibody and the capture antibody is an antibody which recognizes an amino acid sequence from position 151 to position 244 in an amino acid sequence represented by SEQ ID NO. 1, and
   the other of the detection antibody and the capture antibody is an antibody which recognizes a phosphorylated epitope including a phosphorylated amino acid residue in the amino acid sequence represented by SEQ ID NO. 1;
   a step of estimating, based on intensity of the signal, an amount of a tau protein accumulated in a brain of the subject which amount is quantified by tau PET; and
   a step of obtaining information on whether or not the subject, from whom/which the biological sample has been collected, is at Braak stage I/II or Braak stage III or higher, based on the amount of the tau protein accumulated in the brain of the subject which amount has been estimated, wherein
   the intensity of the signal does not correlate with an amount of amyloid accumulated in the brain of the subject which amount is quantified by amyloid PET.

### Advantageous Effects of Invention

In an aspect of the present invention, it is possible to realize a method, a device, etc. each of which makes it possible to estimate, in a simple manner, the amount of a tau protein accumulated in the brain.

### BRIEF DESCRIPTION OF DRAWINGS

FIG. 1 shows the configuration of a main part of an estimating device in accordance with an aspect of the present invention.

FIG. 2 shows the configuration of a main part of an information obtaining device in accordance with an aspect of the present invention.

FIG. 3 is a schematic view of a phosphorylated tau protein and an antibody set which was used in measurement methods of an example and a comparative example.

FIG. 4 shows brain sites corresponding to the Braak stages.

FIG. 5 shows the relationship between the concentrations of a phosphorylated tau protein which were obtained by the measurement method of the comparison example and SUVR values in tau PET images.

FIG. 6 shows the relationship between the concentrations of the phosphorylated tau protein which were obtained by the measurement method of the example and the SUVR values in the tau PET images.

FIG. 7 is an image in which it was investigated at which sites in the cerebrum and how strongly the concentrations of blood p-Tau181 which had been measured by the measurement methods of the example and the comparative example correlated with the amounts of amyloid and tau deposited in the cerebrum which had been respectively quantified by amyloid PET and tau PET.

FIG. 8 shows a result of evaluating whether or not it was possible to discriminate between Braak stage I/II and Braak stage III or higher, based on the concentrations of the phosphorylated tau protein which had been obtained by the measurement method of the example.

FIG. 9 shows the relationship between the concentrations of the phosphorylated tau protein which were obtained by the measurement method of the example and the SUVR values and AD tau scores in the tau PET images.

FIG. 10 shows the relationship between the concentrations of the phosphorylated tau protein which were obtained by the measurement method of the comparative example and the SUVR values and the AD tau scores in the tau PET images.

### DESCRIPTION OF EMBODIMENTS

### 1. Method for Estimating the Amount of a Tau Protein Accumulated in the Brain of a Subject

A method for estimating the amount of a tau protein accumulated in the brain of a subject (hereinafter may be abbreviated as “estimation method”) in accordance with an aspect of the present invention is a method for estimating the amount of a tau protein accumulated in the brain of a subject, by detecting a phosphorylated tau protein in a biological sample collected from the subject. This estimation method makes it possible to estimate tau pathology in the brain of a subject.

In the present specification, the “tau pathology” refers to the abnormal accumulation of a tau protein, such as a phosphorylated tau protein, which appears in the brain of a patient with a tauopathy and a brain site(s) in which such accumulation occurs. In the present specification, “estimating the amount of a tau protein accumulated in the brain of a subject” refers to estimating the amount of a tau protein, such as a phosphorylated tau protein, accumulated in the brain of a subject. The amount of the tau protein accumulated in the brain may be the amount of the tau protein accumulated in the cerebral cortex in which the accumulation of the tau protein is observed in Alzheimer's disease.

The phosphorylated tau protein is a tau protein in which at least one amino acid residue is phosphorylated. Examples of the tau protein includes a human-derived amino acid sequence represented by SEQ ID NO: 1 (NCBI Reference Sequence: NP_005901.2).

For example, the phosphorylation site of the phosphorylated tau protein includes at least one serine residue selected from the group consisting of serine residues at positions 198, 199, 202, 208, 210, 214, 235, 262, 356, 396, 400, 404, 409, 412, 413, 416, and 422 and/or at least one threonine residue selected from the group consisting of threonine residues at positions 181, 205, 212, 217, and 231 in the amino acid sequence of SEQ ID NO: 1.

Examples of the subject include mammals, birds, reptiles, and amphibians. Among others, mammals are preferable. Examples of the mammals include humans and non-human animals. Examples of the non-human animals include: livestock such as cattle, horses, pigs, and sheep; and companion animals or laboratory animals such as dogs, cats, rats, mice, hamsters, monkeys, and rabbits. Preferable are humans. Examples of the birds include poultry such as chickens, wild ducks, and domestic ducks.

Additionally, examples of the subject may include subjects suspected of having mild cognitive impairment or dementia. Furthermore, the subject may be of a certain age group, regardless of whether the subject has mild cognitive impairment or dementia. For example, in a case where the subject is a human, the subject may be any individual who is 30 years old or over, 40 years old or over, 50 years old or over, 60 years old or over, 70 years old or over, or 80 years old or over.

The biological sample collected from the subject is not particularly limited, provided that the biological sample is a sample which can contain the phosphorylated tau protein. Examples of the biological sample include blood samples, saliva, spinal fluid, and urine. The blood samples include whole blood, blood plasma, and blood serum. The blood samples are preferably blood plasma or blood serum, and more preferably blood plasma. Blood plasma can be separated from whole blood collected with a heparin salt (preferably sodium salt), a citrate (preferably sodium salt), or an ethylenediamine tetraacetic acid (EDTA) salt (preferably sodium salt or potassium salt). Blood plasma is more preferably blood plasma separated from whole blood collected with an EDTA salt or a heparin salt.

The estimation method in accordance with an aspect of the present invention includes a preparing step, a detecting step, and an estimating step. Each step is described below.

### (Preparing Step)

In the preparing step, an immune complex of the phosphorylated tau protein in the biological sample from the subject, a capture antibody, and a detection antibody is formed on a capture bead in the presence of a non-capture bead to prepare a measurement sample containing the non-capture bead and the capture bead to which the immune complex binds.

### <Antibodies>

One of the detection antibody and the capture antibody is an antibody which recognizes the amino acid sequence from position 151 to position 224 in the amino acid sequence represented by SEQ ID NO. 1. The antibody which recognizes the amino acid sequence from position 151 to position 224 in the amino acid sequence represented by SEQ ID NO: 1 means an antibody which recognizes an epitope that is present in the amino acid sequence from position 151 to position 224 in the amino acid sequence represented by SEQ ID NO. 1. This antibody is an antibody which recognizes the amino acid sequence of a middle part of the tau protein. Examples of the antibody include Tau Monoclonal Antibody (clone: HT7, TAU-5) available from Thermo Fisher Scientific Inc. The antibody is an antibody which recognizes, in the amino acid sequence represented by SEQ ID NO: 1, preferably the amino acid sequence from position 151 to position 210 (the epitope is present in the amino acid sequence from position 151 to position 210), more preferably the amino acid sequence from position 151 to position 200, and even more preferably the amino acid sequence from position 151 to position 170, because it is possible to more accurately estimate tau pathology in the brain of the subject.

The other of the detection antibody and the capture antibody is an antibody which recognizes a phosphorylated epitope including the phosphorylated amino acid residue in the amino acid sequence represented by SEQ ID NO. 1 (hereinafter may be referred to as “p-tau-specific antibody”). The phosphorylated amino acid residue includes at least one phosphorylated serine residue selected from the group consisting of phosphorylated serine residues at positions 198, 199, 202, 208, 210, 214, 235, 262, 356, 396, 400, 404, 409, 412, 413, 416, and 422 and/or at least one phosphorylated threonine residue selected from the group consisting of phosphorylated threonine residues at positions 181, 205, 212, 217, and 231 in the amino acid sequence of SEQ ID NO: 1. The phosphorylated epitope recognized by the p-tau-specific antibody preferably includes at least one phosphorylated serine residue selected from the group consisting of phosphorylated serine residues at positions 199, 202, 214, and 262 and/or at least one phosphorylated serine residue selected from the group consisting of phosphorylated serine residues at positions 181, 205, 212, 217, and 231, because it is possible to detect a partially C-terminally deleted phosphorylated tau protein.

As each of the detection antibody and the capture antibody, any of a polyclonal antibody, a monoclonal antibody, and fragments thereof (e.g., Fab, F(ab)2, and the like) can be used. Further, the class and subclass of an immunoglobulin is not particularly limited. An antibody selected from an antibody library by a phage display method or the like may be used. Each of the detection antibody and the capture antibody may be a commercially available antibody.

As the p-tau-specific antibody, Phospho-Tau (Thr181) Antibody (clone: 5HCLC), Phospho-Tau (Thr231) Polyclonal Antibody (Cat #: 710561), Phospho-Tau (Ser214) Polyclonal Antibody (Cat #: 44-742G), or the like available from Thermo Fisher Scientific Inc. can be, for example, used.

In the present specification, the capture antibody is an antibody which binds to the tau protein and is immobilized on a bead described later. Immobilization on the bead may be indirect immobilization mediated by a substance such as avidin (or streptavidin) or biotin. In the present specification, the detection antibody is an antibody which binds to the phosphorylated tau protein and preferably has a labeling substance described later.

### <Beads>

Beads are not limited, provided that the beads have a property that the beads can be spatially separated from each other. The beads may be provided in such a form that the beads can be spatially separated in a plurality of locations (e.g., wells). For example, the beads may have a shape of a sphere, a disk, a ring, a cube, or the like. The beads may be fine particles (e.g., a plurality of particles suspended in a liquid), nanotubes, or the like.

The size or shape of the beads can be designed as appropriate. For example, the shape of the beads includes a sphere, a cube, an ellipsoid, and a tube. The average diameter (in the case of a substantially spherical shape) or the average maximum cross-sectional size (in the case of another shape) of the beads may be not less than approximately 0.1 μm, not less than approximately 1 μm, not less than approximately 10 μm, not less than approximately 100 μm, or not less than approximately 1 mm. The average diameter or maximum external size of the beads may be approximately 0.1 μm to approximately 100 μm, may be approximately 1 μm to approximately 100 μm, may be approximately 10 μm to approximately 100 μm, may be approximately 0.1 μm to approximately 1 mm, may be approximately 1 μm to approximately 10 mm, or may be approximately 0.1 μm to approximately 10 μm. The average diameter or average maximum cross-sectional size of the beads used in the present embodiment is an arithmetic average value of the diameters/maximum cross-sectional sizes of the beads.

The average maximum cross-sectional size of the beads can be measured with the use of, for example, laser light scattering, microscopy, a sieve analysis, an electric resistance method, or another known technique. The average diameter of the beads is a volume-based median diameter measured by a particle size distribution measurement device employing a laser diffraction and scattering method. Examples of such a particle size distribution measurement device include “MICROTRAC MT3000II” available from NIKKISO CO., LTD.

The beads are insoluble or substantially insoluble in a solvent or a solution used in measurement. The beads may be, for example, porous, substantially porous, hollow, or partially hollow. The beads are preferably non-porous solids or substantially non-porous solids (e.g., essentially pore-free solids). The beads may substantially absorb or absorb the solvent. Preferably, the beads do not absorb or do not substantially absorb the solvent.

The material of the beads can be selected from: plastics and synthetic polymers (e.g., polyethylene, polypropylene, polystyrene, polyamide, polyurethane, phenolic polymer, nitrocellulose, and the like); naturally derived polymers (latex rubber, polysaccharide-supported metal, and metal compounds (e.g., metal compounds including gold, silver, steel, aluminum, copper, and the like)); inorganic glass; silica; mixtures thereof; and the like. The beads preferably include a magnetic material. In addition, the beads is preferably capable of being optically detected.

The beads may be commercially available beads. For example, carboxyl-functionalized paramagnetic beads (diameter: 2.7 μm) available from Agilent Technologies, Inc. can be used.

In terms of, for example, the stabilization of an antigen-antibody reaction, a buffer solution (buffer) used for the beads is preferably a buffer solution to which a chelating agent is added, and more preferably a buffer solution to which EDTA is added. The addition of EDTA makes it possible to suppress a measurement error occurrence rate. In terms of the suppression of the measurement error occurrence rate (in particular, the measurement error occurrence rate resulting from the measurement becoming impossible due to the aggregation of the beads), the chelating agent is preferably added to the buffer solution used for the capture bead.

A buffer solution with which the biological sample is diluted (sample buffer) preferably contains a blocking agent or a blood serum component derived from a mammal, in terms of improvement in sensitivity of the antigen-antibody reaction and reduction in background during the detection of the antigen-antibody reaction. Examples of the blocking agent and the blood serum component derived from an mammal include albumin, casein, and skim milk.

In the present specification, the capture bead is a bead on which the complex including the phosphorylated tau protein can be immobilized. For example, the capture bead can be a bead on which a molecule that can bind to the phosphorylated tau protein (hereinafter also referred to as “capture molecule”) is directly or indirectly immobilized. As the capture molecule, the capture antibody described above is exemplified.

A method for preparing the capture bead is not limited, provided that the capture molecule can be bound to the bead. For example, the capture bead may be prepared with the use of a commercially available kit such as Simoa (trademark) Homebrew Assay Development kit available from Quanterix Corporation. In order to suppress non-specific binding, the capture bead may be blocked with albumin, casein, skim milk, or the like.

In the present specification, the non-capture bead is a bead which does not substantially bind to the phosphorylated tau protein. The non-capture bead does not have a capture molecule. The material of the bead used as the non-capture bead may be the same as or different from that of the bead used as the capture beads. As the non-capture bead, Dye-Encoded Helper Beads available from Quanterix Corporation may be, for example, used. In order to suppress non-specific binding, the non-capture bead may be blocked with albumin, casein, skim milk, or the like.

A method for forming the immune complex of the phosphorylated tau protein in the biological sample, the capture antibody, and the detection antibody on the capture bead is not limited, provided that the present embodiment is realized. For example, the immune complex can be formed as follows. First, the phosphorylated tau protein in the biological sample is bound to the capture antibody so that the phosphorylated tau protein is bound to (preferably, captured by) the capture bead. Next, the detection antibody is bound to the phosphorylated tau protein captured by the capture antibody. In this manner, the immune complex is formed.

Alternatively, as the method for forming the immune complex, the immune complex of the phosphorylated tau protein in the biological sample, the capture antibody, and the detection antibody may be formed in a buffer solution, and then bound to the capture bead. For example, the bead to which avidin or streptavidin is immobilized is brought into contact with the complex of the biotin-labeled capture antibody, the phosphorylated tau protein, and the detection antibody. This makes it possible to immobilize the immune complex on the capture bead.

The immune complex is formed in the presence of the non-capture bead. The ratio between the numbers of capture beads and non-capture beads when the measurement sample is prepared can be adjusted such that, in a case where the capture beads are regarded as 1, the non-capture beads are not less than approximately 1.5, preferably approximately 1.5 to approximately 9, more preferably approximately 4 to approximately 9, and most preferably approximately 4. In another aspect, the ratio between the numbers of capture beads and non-capture beads when the measurement sample is prepared is such that, in a case where the capture beads are regarded as 1, the non-capture beads are not less than 1.5, preferably 1.5 to 9, more preferably 4 to 9, and most preferably 4.

The total number of beads mixed with the biological sample or the diluted biological sample can be, for example, approximately 100,000 to 1,000,000, and preferably approximately 400,000 to 800,000 beads, with respect to 100 μl of the biological sample or the diluted biological sample.

The preparation of the measurement sample may be carried out in the buffer solution (e.g., 1 to 5×PBS) in which the immune complex can be formed. During the preparation of the measurement sample, the capture bead may be washed with a buffer solution after the phosphorylated tau protein in the biological sample is captured by the capture antibody and before the detection antibody is bound. The capture bead may also be washed with a buffer solution after the immune complex of the phosphorylated tau protein in the biological sample, the detection antibody, and the capture antibody is formed on the capture bead.

The detection antibody may be added such that the final concentration thereof becomes 0.05 μg/mL to 1 μg/mL (preferably, approximately 0.1 μg/mL to 0.5 μg/mL) with respect to 100 μL of the biological sample or the diluted biological sample. The detection antibody preferably has a labeling substance.

### (Detecting Step)

In the detecting step, a signal derived from the immune complex in the measurement sample is detected.

The detection of the signal derived from the immune complex in the measurement sample can be carried out by detecting a labeling substance included in the immune complex. The labeling substance is not particularly limited, provided that a signal which can be detected is generated. The labeling substance may be, for example, a substance which generates a signal itself (hereinafter also referred to as “signal generating substance”) or may be a substance which catalyzes the reaction of another substance so that a signal is generated. Examples of the signal generating substance include fluorescent substances and radioactive isotopes. Examples of the substance which catalyzes the reaction of another substance so that a signal which can be detected is generated include enzymes. Examples of the enzymes include alkaline phosphatase, peroxidase, β-galactosidase, and luciferase. Examples of the fluorescent substances include fluorescent dyes such as fluorescein isothiocyanate (FITC), rhodamine, and Alexa Fluor (registered trademark) and fluorescent proteins such as GFP. Examples of the radioactive isotopes include 125I, 14C, and 32P. Among them, as the labeling substance, an enzyme is preferable, and β-galactosidase is particularly preferable.

The substrate of the enzyme can be selected, as appropriate, from known substrates in accordance with the type of the enzyme. For example, in a case where alkaline phosphatase is used as the enzyme, examples of the substrate include: chemiluminescent substrates such as CDP-Star (registered trademark) (disodium 4-chloro-3-(methoxyspiro[1,2-dioxetane-3,2′-(5′-chloro)tricyclo[3.3.1.13,7]decan]-4-yl)phenyl phosphate) and CSPD (registered trademark) (disodium 3-(4-methoxyspiro[1,2-dioxetane-3,2′-(5′-chloro)tricyclo[3.3.1.13,7]decan]-4-yl)phenyl phosphate); and chromogenic substrates such as 5-bromo-4-chloro-3-indolyl phosphate (BCIP), disodium 5-bromo-6-chloro-indolyl phosphate, and p-nitrophenyl phosphate. In a case where β-galactosidase is used as the enzyme, resorufin β-D-galactopyranoside can be used. In a case where alkaline phosphatase is used as the enzyme, examples of the substrate include: chemiluminescent substrates such as CDP-Star (registered trademark) (disodium 4-chloro-3-(methoxyspiro[1,2-dioxetane-3,2′-(5′-chloro)tricyclo[3.3.1.13,7]decan]-4-yl)phenyl phosphate) and CSPD (registered trademark) (disodium 3-(4-methoxyspiro[1,2-dioxetane-3,2′-(5′-chloro)tricyclo[3.3.1.13,7]decan]-4-yl)phenyl phosphate); and chromogenic substrates such as 5-bromo-4-chloro-3-indolyl phosphate (BCIP), disodium 5-bromo-6-chloro-indolyl phosphate, and p-nitrophenyl phosphate. In a case where β-galactosidase is used as the enzyme, resorufin β-D-galactopyranoside can be used. In cases where peroxidase is used as the enzyme, Super Signal ELISA series available from Thermo Fisher Scientific Inc. can be, for example, used. These enzyme reactions are preferably carried out before the bead is placed in a microwell described later, but may be carried out after the bead is placed in the microwell.

The detection antibody may be biotinylated, and then bound to avidin or streptavidin each of which is labeled with the labeling substance. Thereafter, the signal may be detected.

The detection of the signal can be carried out with respect to all or some of the beads which are contained in the measurement sample and which include the capture bead and the non-capture bead. The detection of the signal is preferably carried out with respect to each of the beads. Note, here, that the capture bead differs from the non-capture bead in presence or absence, intensity, wavelength, and the like of the signal. Therefore, it is preferable to use a detecting device which can distinguish these.

As a method for detecting the signal with respect to each of the beads, a flow cytometer may be, for example, used to detect the signal, in a case where the signal is fluorescence. Alternatively, the measurement sample may be brought into contact with a substrate having a plurality of microwells, the capture bead and the non-capture bead in the measurement sample may be placed in the microwells, and then the signal may be detected in each of the microwells.

On the substrate, the microwells can be arranged on a substantially flat surface or in a non-planar three-dimensional array. The microwells may be arranged in a regular pattern or may be randomly distributed. The microwells are preferably parts in a regular pattern on the substantially flat surface.

The number of the microwells included in the substrate may be approximately 1,000 to 1,000,000, preferably 10,000 to 500,000, and more preferably approximately 100,000 to 500,000, per measurement sample.

Examples of the material of the substrate include: glass (including modified and/or functionalized glass); plastics (polypropylene, polyethylene, polybutylene, polyurethane, cyclic olefin copolymer (COC), cyclic olefin polymer (COP), Teflon (registered trademark), polysaccharide, nylon, nitrocellulose, and the like); elastomers (e.g., dimethylsiloxane, polyurethane, and the like); composite materials; ceramic; silica and silica-based materials (including silicon and modified silicon); carbon; metal; and fiber optic bundles.

The volume of each of the microwells may be set, as appropriate, in accordance with the size of the beads. For example, the number of beads which are held in a single well is approximately 1 to 5, preferably approximately 1 to 2, and more preferably 1. Therefore, the volume of each of the microwells is preferably such that the above number of beads are held in a single well (e.g., Specification of US Patent Application Publication No. 2011/0212848, International Publication No. WO 2011/109364). More specifically, the volume of each of the microwells falls within the range of 100 aL to 1 pL, preferably 1 fL to 500 fL, and more preferably 10 fL to 90 fL. The plurality of microwells present on a single substrate each preferably have the substantially same volume.

A method for forming the microwells is not limited, provided that the microwells having a desired size can be formed. For example, the microwells may be formed by a known method such as photolithography, a stamping technique, a molding technique, or an etching technique.

The placement of the beads in the microwells can be carried out with the use of a known method. The beads may be placed in all of the microwells on the substrate or may be placed in some of the microwells.

For example, the measurement sample may be brought into contact with the substrate on which the microwells are designed, and then the substrate and the solution are centrifuged so that the beads are placed in the microwells. In a case where the beads are magnetic, a magnet may be used to place the beads in the microwells. In a case where the microwells are formed at an end of a fiber optic bundle, the measurement sample may be brought into contact with the other ends and their vicinities of the optical fibers, and then a force such as a centrifugal force or a pressure may be applied so that the beads are placed in the microwells.

After the beads are placed in the microwells, the measurement sample remaining on the substrate around the microwells may be removed, and the surface of the substrate may be washed and/or wiped out so that the excess measurement sample is removed. As the substrate including the microwells, Simoa (trademark) Disc available from Quanterix Corporation may be, for example, used.

The microwells after the beads are placed therein may be sealed with a fluorous liquid, an oil (e.g., a mineral oil or a fluorinated oil), a ferromagnetic fluid, a nonaqueous polymer solution (e.g., a thickener), or the like. For example, Simoa (trademark) Sealing Oil available from Quanterix Corporation may be used for sealing.

In a case where the labeling substance is an enzyme or a fluorescent dye, the detection with respect to the beads placed in the microwells may be carried out, for example, by capturing an image of the signal with a CCD camera or the like. In a case where the labeling substance is a radioactive isotope, the detection may be carried out with the use of autoradiography.

The beads placed in the microwells are each the capture bead or the non-capture bead. The capture bead which has captured the phosphorylated tau protein emits the signal derived from the labeling substance (i.e., the signal derived from the immune complex). On the contrary, the non-capture bead which has not captured the target protein does not emit the signal derived from the labeling substance. Thus, the target protein can be quantified by counting the number of wells in each of which the signal derived from the labeling substance is emitted. The microwells in each of which the detection of the signal is carried out may be all of the microwells on the substrate or may be some of the microwells.

The number of the wells in each of which the signal derived from the labeling substance is emitted can be measured, for example, by Simoa (trademark) HD-1 Analyzer and Simoa (trademark) HD-X Analyzer each available from Quanterix Corporation. In a case where Simoa (trademark) HD-1 Analyzer and Simoa (trademark) HD-X Analyzer are used, the intensity of the signal is represented by an average enzymes per bead (AEB) value. Thus, the AEB value may be used as the intensity of the signal derived from the immune complex in the measurement sample.

The AEB value may be converted into a measured value of the phosphorylated tau protein with the use of a calibration curve. Note, here, that the measured value of the phosphorylated tau protein refers to a value reflecting the amount or concentration of the phosphorylated tau protein. In a case where the measured value is expressed as the “amount”, the measured value may be expressed in terms of moles or mass, but is preferably expressed in terms of mass. In a case where the value is expressed as the “concentration”, the measured value may be expressed in terms of a molar concentration or the ratio (mass/volume) of mass to a constant volume of the biological sample, but is preferably mass/volume.

In the estimation method in accordance with an aspect of the present invention, it is preferable to use β-galactosidase as the labeling substance and use resorufin β-D-galactopyranoside as the substrate, in accordance with the protocol provided by the Quanterix Corporation. Further, it is preferable that the beads are applied to Simoa (trademark) Disc and the intensity of the signal is obtained by Simoa (trademark) HD-1 Analyzer and Simoa (trademark) HD-X Analyzer.

The estimation method in accordance with an aspect of the present invention is a sandwich immunoassay method in which a sandwich immune complex containing the phosphorylated tau protein is formed and the phosphorylated tau protein is detected.

### (Estimating Step)

In the estimating step, the amount of the tau protein accumulated in the brain of the subject which is quantified by tau PET is estimated based on the intensity of the signal obtained in the detecting step. The amount of the tau protein accumulated in the brain is represented, for example, by a standardized uptake value ratio (SUVR) value, an AD tau score, or the like in a tau PET image. The AD tau score is an AD tau score disclosed in Movement Disorders, 2022, Vol. 37, No. 11: 2236-2246. The AD tau score is a score which is obtained through machine learning-based measurement and which indicates an AD-related feature of the tau PET image.

In tau PET, it is possible to detect or identify tau pathology with the use of a PET agent (PET tracer) such as pyridinyl-butadienyl-benzothiazole (PBB) 3.

The SUVR indicates the ratio between the degree of accumulation of the PET tracer in a region of the brain in which the degree of accumulation of the PET tracer is low and is small in variation (reference region) and the degree of accumulation of the PET tracer in each site of the brain in which tau pathology is present and the accumulation of the PET tracer is present (region of interests). The SUVR is an index for quantitatively representing the intensity of accumulation in tau pathology detected by PET. Examples of the reference site include the cerebellar cortex and cerebral white matter. Examples of the region of interests include brain sites which correspond to the Braak stages and a specific site which is located in the temporal lobe and in which the accumulation of a tau protein is prominently observed in Alzheimer's disease (temporal meta-ROI). The brain sites which exhibit specific c accumulation in tau pathology include the brain regions which are involved in memory (e.g., the hippocampus and entorhinal cortex) and the neocortical region of the brain.

More specifically, the intensity of the signal obtained in the detecting step is compared with a given reference value. Then, based on this comparison, the amount of the tau protein accumulated in the brain is estimated.

The given reference value refers to a reference value of the intensity of the signal derived from the phosphorylated tau protein. The reference value may be determined based on the intensity of a signal derived from the phosphorylated tau protein in a biological sample of an individual with a tauopathy (positive control value).

The amount of the tau protein accumulated in the brain of the subject may be estimated with the use of the measured value of the phosphorylated tau protein, instead of the intensity of the signal.

In the development of therapeutic agents for Alzheimer's disease, it is an important evaluation criterion whether the accumulation of an abnormal tau protein in the brain, which is present downstream of the accumulation of amyloid and which is a direct cause of a neurodegenerative disorder, is reduced. In the case of an agent for an abnormal tau protein in the brain, it is necessary to evaluate the amount of a tau protein accumulated in the brain, in order to determine the direct effect of the agent.

The amount of an abnormal tau protein accumulated in the brain can be quantified by tau PET. However, tau PET is an expensive examination, and has the disadvantage that there is a risk of invasiveness due to radiation. Therefore, there is a demand for the development of body fluid biomarkers which accurately reflect the amount of a tau protein accumulated in the brain that is quantified by tau PET, particularly, blood biomarkers which are low-cost and non-invasive and which can be used in all medical institutions.

For the first time, the inventors of the present invention found that the amount of a tau protein accumulated in the brain which is quantified by tau PET positively correlates with the concentration of a phosphorylated tau protein which is detected by the combination of a p-tau-specific antibody and an antibody which recognizes an epitope that is present in the amino acid sequence from position 151 to position 224 in the amino acid sequence represented by SEQ ID NO. 1. By the estimation method in accordance with an aspect of the present invention, it is possible to estimate, in a simple manner without the use of tau PET, the amount of a tau protein accumulated in the brain, and possible to obtain information which assists in determining whether or not a subject is affected by a tauopathy. The amount of a phosphorylated tau protein which is detected by the method can be used as an index for estimating tau pathology in the brain.

The intensity of the signal does not correlate with the amount of amyloid accumulated in the brain of the subject which is quantified by amyloid PET. In amyloid PET, it is possible to measure the amount of amyloid accumulated in the brain with the use of a PET agent such as PiB (Pittsburgh Compound B).

In a case where an anti-amyloid drug such as lecanemab is administered to a target, the amount of amyloid accumulated in the brain of the target decreases. In a conventional method for measuring a phosphorylated tau protein in a biological sample (e.g., a measurement method in a comparative example), a fragment of a phosphorylated tau protein which is secreted or released into the blood and the like in dependence on the accumulation of amyloid in the brain is detected. Thus, in the conventional method for measuring a phosphorylated tau protein, the measured value of a phosphorylated tau protein in a biological sample decreases after the administration of an anti-amyloid drug. Therefore, by the conventional method for measuring a phosphorylated tau protein in a biological sample, it has not been able to determine whether or not the accumulation of abnormal tau in the brain which is induced by amyloid has been reduced by the administration of an anti-amyloid drug.

For the first time, the inventors of the present invention found that the amount of amyloid accumulated in the brain which is measured by amyloid PET does not correlate with the concentration of a phosphorylated tau protein which is detected, as in the measurement method of the example, by the combination of a p-tau-specific antibody and an antibody which recognizes an epitope that is present in the amino acid sequence from position 151 to position 224 in the amino acid sequence represented by SEQ ID NO. 1. The measurement method makes it possible to measure a fragment of a phosphorylated tau protein which is secreted or released into the blood and the like in response to the accumulation of a tau protein in the brain (for example, a blood phosphorylated tau protein which is independent of the accumulation of amyloid in the brain), and makes it possible to provide a biomarker which purely reflects the amount of a tau protein accumulated in the brain without being affected by the accumulation of amyloid (in other words, the interference effect by the accumulation of amyloid). Therefore, the estimation method in accordance with an aspect of the present invention can also be used, for example, in determining the effect of an anti-amyloid drug in actual clinical practice or in determining the selection of the administration of an anti-amyloid drug to a target.

### 2. Method for Obtaining Information which Assists in Determining Whether or not a Subject is at Braak Stage I/II or Braak Stage III or Higher

Aspects of the present invention also encompass a method for obtaining information which assists in determining whether or not a subject is at Braak stage I/II or Braak stage III or higher. In this method, information which assists in determining whether or not a subject is at Braak stage I/II or Braak stage III or higher is obtained based on the amount of a tau protein accumulated in the brain of the subject which has been estimated by the [1. Method for estimating the amount of a tau protein accumulated in the brain of a subject]. Braak stage III or higher indicates Braak stage III/IV or Braak stage V/VI.

The Braak stages are international stage classifications which reflect the pathological spread of the accumulation of a tau protein in Alzheimer's disease. At Braak stage I/II, the accumulation of the tau protein is observed in the entorhinal cortex region of the cerebrum. At Braak stage III/IV, the accumulation of the tau protein is also observed in the limbic system region of the cerebrum, such as the hippocampus, and in the site of the inferior temporal lobe. At Braak stage V/VI, the accumulation of the tau protein which has spread in the neocortical region of the cerebrum is observed. The accumulation of tau in the site corresponding to Braak stage I/II is also observed in individuals with normal aging. However, it is known that the accumulation of the tau protein in patients with Alzheimer's disease reaches Braak stage III/IV or higher.

More specifically, in the method for obtaining information which assists in determining whether or not a subject is at Braak stage I/II or Braak stage III or higher, the estimated amount of the tau protein accumulated in the brain of the subject is compared with a given tau protein accumulation reference value. In a case where the estimated amount of the tau protein accumulated in the brain of the subject is higher than the given tau protein accumulation reference value, information that the subject is at Braak stage III or higher is obtained. The given tau protein accumulation reference value may be determined based on the amount of the tau protein accumulated in the brain of an individual at Braak stage III or higher (positive control value).

The given tau protein accumulation reference value may be a cutoff value set by a receiver operating characteristic curve (ROC) analysis.

### 3. Method for Obtaining Information which Assists in Evaluating the Degree of Progression of a Tauopathy

Aspects of the present invention also encompass a method for obtaining information which assists in determining whether or not a subject is affected by a tauopathy or information which assists in evaluating the degree of progression of a tauopathy (hereinafter may be abbreviated as “information obtaining method”). In the information obtaining method in accordance with an aspect of the present invention, information which assists in determining whether or not a subject is affected by a tauopathy or information which assists in evaluating the degree of progression of a tauopathy is obtained based on the amount of a tau protein accumulated in the brain of the subject which has been estimated by the [1. Method for estimating the amount of a tau protein accumulated in the brain of a subject]. Examples of the tauopathy include Alzheimer's disease, progressive supranuclear palsy, cerebral cortical degeneration, frontotemporal dementia, and Pick's disease.

More specifically, the estimated amount of the tau protein accumulated in the brain of the subject is compared with a given tau protein accumulation reference value. In a case where the estimated amount of the tau protein accumulated in the brain of the subject is higher than the given tau protein accumulation reference value, information that the subject is affected by a tauopathy or that a tauopathy in the subject is progressing is obtained. The given tau protein accumulation reference value may be determined based on the amount t of the tau protein accumulated in the brain of an individual with a tauopathy (positive control value). The given tau protein accumulation reference value may be a cutoff value set by a receiver operating characteristic curve (ROC) analysis. The information obtaining method in accordance with an aspect of the present invention makes it possible to evaluate the degree of progression of a tauopathy without being affected by the accumulation of amyloid.

### 4. Device which Estimates the Accumulation of a Tau Protein in the Brain of a Subject

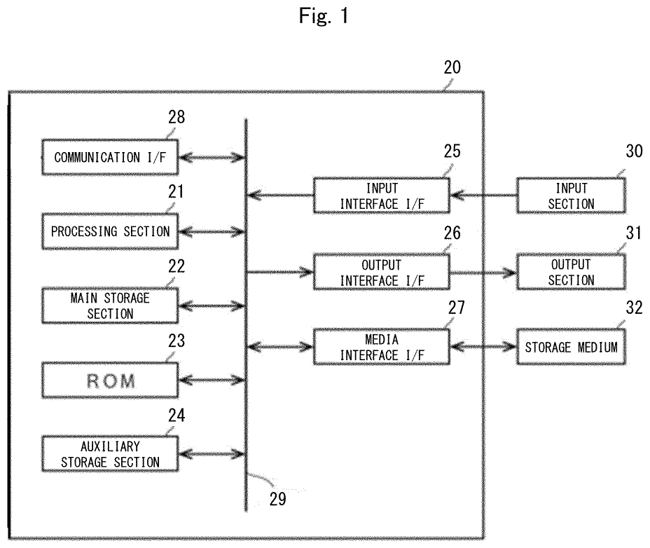

The present invention also relates to a device which estimates the accumulation of a tau protein in the brain of a subject (hereinafter may be abbreviated as “estimating device”). The estimating device in accordance with an embodiment of the present invention is described in detail with reference to FIG. 1. FIG. 1 schematically shows the configuration of an estimating device 20 in accordance with an embodiment of the present invention. The estimating device 20 is a device which estimates the amount of a tau protein accumulated in the brain of a subject by detecting a phosphorylated tau protein in a biological sample collected from the subject.

As shown in FIG. 1, the estimating device 20 is connected to an input section 30, an output section 31, and a storage medium 32. In the estimating device 20, a processing section (CPU) 21, a main storage section 22, a read only memory (ROM) 23, an auxiliary storage section 24, a communication interface (I/F) 28, an input interface (I/F) 25, an output interface (I/F) 26, and a media interface (I/F) 27 are connected to each other by a bus 29 so that data communication can be performed therebetween.

The CPU 21 is a processing section of the estimating device 20. The estimating device 20 functions by the CPU 21 executing a computer program stored in the auxiliary storage section 24 or the ROM 23 and processing obtained data.

The ROM 23 is constituted by a mask ROM, a PROM, an EPROM, an EEPROM (registered trademark), or the like, and a computer program executed by the CPU 21 and data used therefor are recorded in the ROM 23. The CPU 21 may be an MPU 21. In the ROM 23, a boot program executed by the CPU 21 at the time of start of the estimating device 20, a program relating to operation of hardware of the estimating device 20, and settings are stored.

The main storage section 22 is constituted by a random access memory (RAM) such as an SRAM or a DRAM. The main storage section 22 is used to read computer programs recorded in the ROM 23 and the auxiliary storage section 24. The main storage section 22 is also used as a working area in which the CPU 21 executes these computer programs.

The auxiliary storage section 24 is constituted by a hard disk, an optical disk, a semiconductor memory device such as a flash memory, or the like. In the auxiliary storage section 24, stored are (i) various computer programs which are executed by the CPU 21, such as an operating system and an application program, and (ii) various setting data used to execute the computer programs. Specifically, in the auxiliary storage section 24, stored are a measurement program (described later), a reference value of the intensity of a signal, a reference value of a measured value of a phosphorylated tau protein, and the like.

The communication I/F 28 is constituted by a serial interface such as USB, IEEE1394, or RS-232C, a parallel interface such as SCSI, an IDE, or IEEE1284, an analog interface which is constituted by a D/A converter, an A/D converter, or the like, a network interface controller (NIC), or the like. Under control by the CPU 21, the communication I/F 28 receives data from a measurement unit or another external device, and transmits or displays, to or on the measurement unit or the outside, information stored or generated by the estimating device 20, as necessary. The communication I/F 28 may perform communication with the measurement unit or another external device via a network.

The input I/F 25 is constituted by, for example, a serial interface such as USB, IEEE1394, or RS-232C, a parallel interface such as SCSI, an IDE, or IEEE1284, an analog interface which is constituted by a D/A converter, an A/D converter, or the like, or the like. The input I/F 25 accepts a character input, a click, a voice input, or the like performed by the input section 30. The content of an accepted input is stored in the main storage section 22 or the auxiliary storage section 24.

The input section 30 is constituted by a touch panel, a keyboard, a mouse, a pen tablet, a microphone, or the like, and performs a character input or a voice input into the estimating device 20. The input section 30 may be connected to the estimating device 20 from the outside of the measurement device 20, or may be integrated with the estimating device 20.

The output I/F 26 is constituted by, for example, an interface similar to that constituting the input I/F 25. The output I/F 26 outputs, to the output section 31, information generated by the CPU 21. The output I/F 26 outputs, to the output section 31, information generated by the CPU 21 and stored in the auxiliary storage section 24.

The output section 31 is constituted by, for example, a display, a printer, or the like, and displays a measurement result transmitted from the measurement unit and various operating windows, a measurement result, or the like in the estimating device 20.

The media I/F 27 reads, for example, application software or the like stored in the storage medium 32. The read application software or the like is stored in the main storage section 22 or the auxiliary storage section 24. The media I/F 27 also writes, in the storage medium 32, information generated by the CPU 21. The media I/F 27 writes, in the storage medium 32, information generated by the CPU 21 and stored in the auxiliary storage section 24.

The storage medium 32 is constituted by a flexible disk, a CD-ROM, a DVD-ROM, or the like. The storage medium 32 is connected to the media I/F 27 by a flexible disk drive, a CD-ROM drive, a DVD-ROM drive, or the like. In the storage medium 32, for example, an application program for a computer to execute operation may be stored.

The CPU 21 may obtain application software and various settings necessary to control the estimating device 20, via a network instead of reading from the ROM 23 or the auxiliary storage section 24. The application program can be stored in the auxiliary storage section 24 of a server computer on a network, and the estimating device 20 can access the server computer, download the computer program, and store the computer program in the ROM 23 or the auxiliary storage section 24.

In the ROM 23 or the auxiliary storage section 24, for example, an operating system which provides a graphical user interface environment, such as Windows (registered trademark) manufactured and sold by Microsoft Corporation in America, may be installed.

The processing section 21 accepts an input which is performed by an examiner on the input section 30 and which is for obtaining a signal derived from an immune complex regarding a measurement sample that includes a non-capture bead and a capture bead to which the immune complex binds. Next, the processing section 21 obtains the signal which is derived from the immune complex and which has been detected by the measurement unit (described later) or the like. The processing section 21 then stores, in the auxiliary storage section 24 or the like, the intensity of the signal thus obtained.

The estimating device 20 can be a personal computer or the like. The estimating device 20 may be embedded in the measurement unit (described later). The estimating device in accordance with an aspect of the present invention makes it possible to estimate the amount of a tau protein accumulated in the brain without being affected by the interference effect by the accumulation of amyloid.

### 5. System which Estimates the Amount of a Tau Protein Accumulated in the Brain of a Subject

The present invention also relates to a system which estimates the amount of a tau protein accumulated in the brain of a subject. The system which estimates the amount of a tau protein accumulated in the brain of a subject includes an estimating device 20 and a measurement unit. The system which estimates the amount of a tau protein accumulated in the brain of a subject is a system which detects a phosphorylated tau protein in a biological sample collected from a subject and estimates the amount of a tau protein accumulated in the brain of the subject.

The measurement unit includes at least a detecting section including an image capturing device for detecting a signal, such as a CCD camera. In some cases, the measurement unit may further include a sample preparing section for preparing a measurement sample. The detecting section may be a flow cytometer. The detecting section may include a pressurizer, a decompression device, or a centrifuge for placing, in microwells, beads in the measurement sample.

The measurement unit may be integrated with the estimating device 20. Examples of a device which includes the detecting section including the image capturing device and which is integrated with the estimating device 20 include Simoa (trademark) HD-1 Analyzer and Simoa (trademark) HD-X Analyzer.

### 6. Device which Obtains Information for Evaluating the Degree of Progression of a Tauopathy

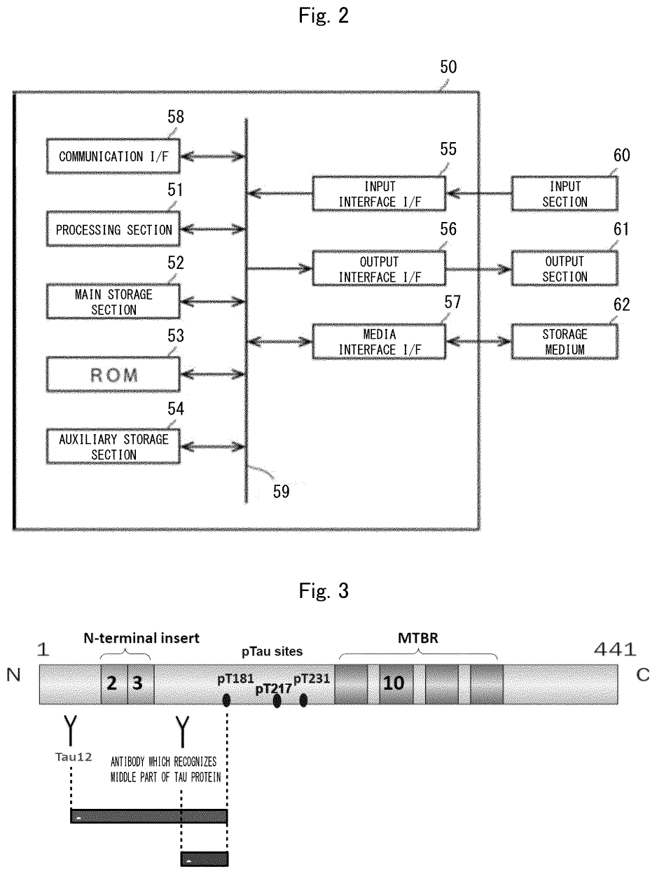

Aspects of the present invention also encompass a device which obtains information that assists in determining whether or not a subject is affected by a tauopathy or information for evaluating the degree of progression of a tauopathy (hereinafter may be abbreviated as “information obtaining device”). FIG. 2 shows the information obtaining device in accordance with an embodiment of the present invention. An information obtaining device 50 is a device which obtains information on whether or not a subject is affected by a tauopathy or information for evaluating the degree of progression of a tauopathy, based on the amount of a tau protein accumulated in the brain of the subject which has been estimated by detecting a phosphorylated tau protein in a biological sample collected from the subject. The information obtaining device in accordance with an aspect of the present invention makes it possible to evaluate the degree of progression of a tauopathy without being affected by the accumulation of amyloid.

As illustrated in FIG. 2, the information obtaining device 50 is connected to an input section 60, an output section 61, and a storage medium 62. In the information obtaining device 50, a processing section (CPU) 51, a main storage section 52, a read only memory (ROM) 53, an auxiliary storage section 54, a communication interface (I/F) 58, an input interface (I/F) 55, an output interface (I/F) 56, and a media interface (I/F) 57 are connected to each other by a bus 59 so that data communication can be performed therebetween.

Details of each configuration in FIG. 2 are similar to those of a corresponding configuration in FIG. 1.

The processing section 51 accepts an input which is performed by an examiner on the input section 60 and which is for obtaining a signal derived from an immune complex regarding a measurement sample that includes a non-capture bead and a capture bead to which the immune complex binds. Next, the processing section 51 obtains the signal which is derived from the immune complex and which has been detected by a measurement unit (described later) or the like. Subsequently, the intensity of the obtained signal is compared with a reference value of the intensity of the signal which reference value is stored in the auxiliary storage section 54 or the like. The processing section 51 estimates, based on this comparison, the amount of a tau protein accumulated in the brain. In a case where the estimated amount of the tau protein accumulated in the brain is lower than a tau pathology reference accumulation amount, the processing section 51 determines that a subject is unlikely to be affected by a tauopathy or that the progression of a tauopathy in the subject has not advanced. The processing section 51 stores, in the auxiliary storage section 54 or the like, this determination result as information on whether or not the subject is affected by a tauopathy or information on the degree of progression of a tauopathy. The processing section 51 may output the result to the output section 61 or store the result in the storage medium 62.

### 7. System which Obtains Information for Evaluating the Degree of Progression of a Tauopathy

Aspects of the present invention also encompass a system which obtains information that assists in determining whether or not a subject is affected by a tauopathy or information for evaluating the degree of progression of a tauopathy (hereinafter may be abbreviated as “information obtaining system”). The information obtaining system includes an information obtaining device 50 and a measurement unit. The information obtaining system is a system which obtains information that assists in determining whether or not a subject is affected by a tauopathy or information for evaluating the degree of progression of a tauopathy, based on the estimated amount of a tau protein accumulated in the brain of the subject. Details of the measurement unit are similar to those of the measurement unit described in [4. System which estimates the amount of a tau protein accumulated in the brain of a subject].

The measurement unit may be integrated with the information obtaining device 50. Examples of a device which includes a detecting section including an image capturing device and which is integrated with the information obtaining device 50 include Simoa (trademark) HD-1 Analyzer and Simoa (trademark) HD-X Analyzer.

[8. Device or system which obtains information that assists in determining whether or not a subject is at Braak stage I/II or Braak stage III or higher]

Aspects of the present invention also encompass a device which obtains information that assists in determining whether or not a subject is at Braak stage I/II or Braak stage III or higher. The configuration of the device may be similar to that of the device which obtains information that assists in determining whether or not a subject is affected by a tauopathy (FIG. 2). In a case where the configuration of the device is the configuration in FIG. 2, a processing section 51 estimates the amount of a tau protein accumulated in the brain. In a case where the estimated amount of the tau protein accumulated in the brain is higher than a tau protein accumulation reference amount, the processing section 51 determines that a subject is at Braak stage III or higher.

Aspects of the present invention also encompass a system which obtains information that assists in determining whether or not a subject is at Braak stage I/II or Braak stage III or higher. The system is a system which obtains information that assists in determining whether or not a subject is at Braak stage I/II or Braak stage III or higher, based on the estimated amount of a tau protein accumulated in the brain of the subject. The configuration of the system may be similar to that of the system which obtains information that assists in determining whether or not a subject is affected by a tauopathy.

### 9. Computer Program for Estimating the Amount of a Tau Protein Accumulated in the Brain of a Subject

The present invention also relates to a computer program for estimating the amount of a tau protein accumulated in the brain of a subject (hereinafter may be abbreviated as “estimation program”). The estimation program is used as a measurement program for controlling operation of an estimating device 20. The estimation program causes a computer to carry out a step of obtaining a signal derived from an immune complex regarding a measurement sample that includes a non-capture bead and a capture bead to which the immune complex binds and a step of estimating, based on the intensity of the signal, the amount of a tau protein accumulated in the brain of a subject.

The measurement program and an information obtainment program may be stored in a storage medium such as a hard disk, a semiconductor memory device such as a flash memory, or an optical disk. A form in which the programs are stored in the storage medium is not limited, provided that an information obtaining device can read the programs. Storage in the storage medium is preferably non-volatile.

### 10. Computer Program for Obtaining Information which Assists in Determining Whether or not Subject is Affected by a Tauopathy

Aspects of the present invention also relates to a computer program for obtaining information which assists in determining whether or not a subject is affected by a tauopathy (hereinafter, may be abbreviated as “information obtainment program”). The information obtainment program is used as a measurement program for controlling operation of an information obtaining device 50. The information obtainment program causes a computer to carry out a step of obtaining a signal derived from an immune complex regarding a measurement sample that includes a non-capture bead and a capture bead to which the immune complex binds. The information obtainment program then causes the computer to carry out a step of obtaining information on whether or not a subject, from whom/which a biological sample has been collected, is affected by a tauopathy, based on the estimated amount of a tau protein accumulated in the brain of the subject.

### 11. Computer Program for Obtaining Information which Assists in Determining Whether or not a Subject is at Braak Stage I/II or Braak Stage III or Higher

Aspects of the present invention also encompass a computer program for obtaining information which assists in determining whether or not a subject is at Braak stage I/II or Braak stage III or higher. The program is used as a measurement program for controlling operation of a device which obtains information that assists in determining whether or not a subject is at Braak stage I/II or Braak stage III or higher. The information obtainment program causes a computer to carry out a step of obtaining a signal derived from an immune complex regarding a measurement sample that includes a non-capture bead and a capture bead to which the immune complex binds. The information obtainment program then causes the computer to carry out a step of obtaining information on whether or not a subject, from whom/which a biological sample has been collected, is at Braak stage I/II or Braak stage III or higher, based on the estimated amount of a tau protein accumulated in the brain of the subject.

### 12. Reagent for Detecting a Phosphorylated Tau Protein And Kit Including Such a Detection Reagent

Aspects of the present invention also encompass a reagent for detecting a phosphorylated tau protein (hereinafter may be abbreviated as “detection reagent”). The detection reagent in accordance with an aspect of the present invention is used in the [1. Method for estimating the amount of a tau protein accumulated in the brain of a subject], and contains the above-described capture bead and the above-described non-capture bead. The preferable ratio of the numbers of non-capture beads and capture beads contained in the detection reagent is as described in the [1. Method for estimating the amount of a tau protein accumulated in the brain of a subject].

The detection reagent in accordance with an aspect of the present invention is a reagent for estimating the amount of a tau protein accumulated in the brain of a subject. The reagent contains a detection antibody and a capture antibody. One of the detection antibody and the capture antibody is an antibody which recognizes the amino acid sequence from position 151 to position 244 in the amino acid sequence represented by SEQ ID NO. 1, and the other is a p-tau-specific antibody.

Aspects of the present invention also encompass a reagent kit for detecting a phosphorylated tau protein (hereinafter may be abbreviated as “reagent kit”). The reagent kit in accordance with an aspect of the present invention is used in the [1. Method for estimating the amount of a tau protein accumulated in the brain of a subject], and includes the above-described detection reagent. The reagent kit in accordance with an aspect of the present invention is a reagent kit for estimating the amount of a tau protein accumulated in the brain of a subject, and includes the detection reagent.

The reagent kit may further include a detection antibody, and may include another reagent, such as a blocking agent including albumin, casein, and skim milk and a buffer solution used for suspension of beads and dilution of a sample (buffer; may contain the blocking agent and EDTA), an instrument, and the like. In a case where a capture antibody is not immobilized to a bead, the reagent kit may further include a reagent which contains the capture antibody. In the reagent kit, a plurality of different reagents may be mixed together at respective appropriate volumes and/or in an appropriate form. The reagent kit may provide the plurality of different reagents in respective different vessels. The reagent kit may include instructions which show, for example, a procedure for measuring a phosphorylated tau protein. The instructions may be written or printed on paper or another medium. Alternatively, the instructions may be written in a magnetic tape or an electronic medium which is readable by a computer or the like, such as a disk or a CD-ROM.

Aspects of the present invention also encompass a kit which includes the above-described detection reagent and which is for estimating the amount of a tau protein accumulated in the brain of a subject. The kit may include a computer-readable recording medium in which a computer program for estimating the amount of a tau protein accumulated in the brain of a subject is stored. Examples of the recording medium include hard disks, semiconductor memory devices such as a flash memory, and optical disks.

Aspects of the present invention also encompass a kit which includes the above-described detection reagent and which is for obtaining information that assists in determining whether or not a subject is affected by a tauopathy. The kit may include a computer-readable recording medium in which a computer program for obtaining information that assists in determining whether or not a subject is affected by a tauopathy is stored.

Aspects of the present invention also encompass a kit which includes the above-described detection reagent and which is for obtaining information that assists in determining whether or not a subject is at Braak stage I/II or Braak stage III or higher. The kit may include a computer-readable recording medium in which a computer program for obtaining information that assists in determining whether or not a subject is at Braak stage I/II or Braak stage III or higher is stored.

Aspects of the present invention also encompass a kit which includes the above-described detection reagent and which is for obtaining information for evaluating the degree of progression of a tauopathy. The kit may include a computer-readable recording medium in which a computer program for obtaining information for evaluating the degree of progression of a tauopathy is stored. The degree of progression of a tauopathy can be evaluated, for example, by a Braak stage, an AD tau score, and the like.

### 13. Method for Diagnosing a Tauopathy

Aspects of the present invention also encompass a method for diagnosing a tauopathy. This diagnosing method includes a diagnosing step of diagnosing whether or not a subject is affected by a tauopathy, based on the amount of a tau protein accumulated in the brain which has been estimated by the [1. Method for estimating the amount of a tau protein accumulated in the brain of a subject]. The diagnosing method encompasses a diagnosis conducted by a physician or another qualified medical professional.

In the diagnosing step, the estimated amount of the tau protein accumulated in the brain is compared with a given tau protein accumulation reference value. In a case where the estimated amount of the tau protein accumulated in the brain is higher than the given tau protein accumulation reference value, the subject is diagnosed as being affected by a tauopathy. The “given tau protein accumulation reference value” is as described in [3. Method for obtaining information which assists in determining whether or not a subject is affected by a tauopathy].

The amount of the tau protein accumulated in the brain of the subject may be estimated with the use of a measured value of a phosphorylated tau protein, instead of the intensity of a signal, and then whether or not the subject is affected by a tauopathy may be diagnosed.

By diagnosing whether or not a subject is affected by a tauopathy, it is possible to objectively observe and evaluate the progression of a disease, the progression of a treatment, or the effect of the treatment.

### 14. Method for Diagnosing a Braak Stage

Aspects of the present invention also encompass a method for diagnosing a Braak stage. This diagnosing method includes a diagnosing step of diagnosing whether or not a subject is at Braak stage I/II or Braak stage III or higher, based on the amount of a tau protein accumulated in the brain which has been estimated by the [1. Method for estimating the amount of a tau protein accumulated in the brain of a subject]. The diagnosing method encompasses a diagnosis conducted by a physician or another qualified medical professional.

In the diagnosing step, the estimated amount of the tau protein accumulated in the brain is compared with a given tau protein accumulation reference value. In a case where the estimated amount of the tau protein accumulated in the brain is higher than the given tau protein accumulation reference value, the subject is diagnosed as being at Braak stage III or higher. The “tau protein accumulation reference value” is as described in [2. Method for obtaining information which assists in determining whether or not a subject is at Braak stage I/II or Braak stage III or higher].

The amount of the tau protein accumulated in the brain of the subject may be estimated with the use of a measured value of a phosphorylated tau protein, instead of the intensity of a signal, and then whether or not the subject is at Braak stage I/II or Braak stage III or higher may be diagnosed.

By diagnosing the Braak stage of a subject, it is possible to diagnose or select a patient in whom the onset of dementia is imminent. Moreover, by diagnosing the Braak stage of a subject, it is possible to select a patient for whom the effect of an anti-amyloid drug can be most expected.

### 15. Method for Assisting in Diagnosing a Patient

Aspects of the present invention also encompass a method for assisting in diagnosing a patient. An aspect of the method for assisting in diagnosing a patient includes a step of obtaining data that suggests whether or not a biological sample is a biological sample derived from a patient affected by a tauopathy, based on the amount of a tau protein accumulated in the brain of a patient which has been estimated by the [1. Method for estimating the amount of a tau protein accumulated in the brain of a subject]. An aspect of the method for assisting in diagnosing a patient includes a step of obtaining data that suggests whether or not a biological sample is a biological sample derived from a patient at Braak stage III or higher, based on the amount of a tau protein accumulated in the brain of a patient which has been estimated by the [1. Method for estimating the amount of a tau protein accumulated in the brain of a subject].

### 16. Method for Evaluating the Degree of Progression of a Tauopathy

Aspects of the present invention also encompass a method for determining the degree of progression of a tauopathy. An aspect of the method for evaluating the degree of progression of a tauopathy includes a step of evaluating the degree of progression of a tauopathy of a subject, based on the amount of a tau protein accumulated in the brain of the subject which has been estimated by the [1. Method for estimating the amount of a tau protein accumulated in the brain of a subject].

Aspects of the present invention also encompass a method for estimating a stage classification which represents the degree of progression of a tauopathy. Examples of the stage classification which represents the degree of progression of a tauopathy include the Braak stages and the AD tau scores.

### 17. Method for Assisting in Selecting a Target to which an Anti-Amyloid Drug is to be Administered

Aspects of the present invention also encompass a method for assisting in selecting a target to which an anti-amyloid drug is to be administered. An aspect of the method for assisting in selecting a target to which an anti-amyloid drug is to be administered includes a step of selecting a target to which an anti-amyloid drug is to be administered, based on the amount of a tau protein accumulated in the brain of a target which has been estimated by the [1. Method for estimating the amount of a tau protein accumulated in the brain of a subject].

The target is a target affected by a tauopathy or a target at risk of being affected by a tauopathy. A signal detected in the detecting step of the [1. Method for estimating the amount of a tau protein accumulated in the brain of a subject] positively correlates with the amount of a tau protein accumulated in the brain of a subject which is quantified by tau PET. However, the signal does not correlate with the amount of amyloid accumulated in the brain of the subject which is quantified by amyloid PET. Therefore, since the signal purely reflects the amount of the tau protein accumulated in the brain, it is possible to evaluate the degree of accumulation of the tau protein in the brain before the administration of an anti-amyloid drug, and possible to select a target to which the anti-amyloid drug is to be administered. For example, in a case where the estimated amount of a tau protein accumulated in the brain of a subject is higher than a given tau protein accumulation reference value, the efficacy of an anti-amyloid drug cannot be expected, and, therefore, the subject can be excluded from a target to which the anti-amyloid drug is to be administered.

By the method for assisting in selecting a target to which an anti-amyloid drug is to be administered, it is possible to evaluate whether or not the accumulation of abnormal tau in the brain which is induced by amyloid has been reduced by the administration of an anti-amyloid drug, regarding a target to which the anti-amyloid drug has already been administered. This makes it possible to determine whether or not the target is selected as a target to which the anti-amyloid drug is to be continuously administered or re-administered.

Examples of the anti-amyloid drug include lecanemab and aducanumab.

### 18. Summary of Embodiments

Embodiments of the present invention include, for example, aspects described below.
<1> A method for estimating an amount of a tau protein accumulated in a brain of a subject, the method including: a preparing step of forming, on a capture bead, an immune complex of a phosphorylated tau protein in a biological sample collected from a subject, a capture antibody, and a detection antibody in the presence of a non-capture bead to prepare a measurement sample containing the non-capture bead and the capture bead to which the immune complex binds; a detecting step of detecting a signal derived from the immune complex in the measurement sample; and an estimating step of estimating, based on intensity of the signal, an amount of a tau protein accumulated in a brain of the subject which amount is quantified by tau PET, wherein the non-capture bead does not bind to the immune complex, one of the detection antibody and the capture antibody is an antibody which recognizes an amino acid sequence from position 151 to position 244 in an amino acid sequence represented by SEQ ID NO. 1, the other of the detection antibody and the capture antibody is an antibody which epitope including a recognizes a phosphorylated phosphorylated amino acid residue in the amino acid sequence represented by SEQ ID NO. 1, and the intensity of the signal does not correlate with an amount of amyloid accumulated in the brain of the subject which amount is quantified by amyloid PET.
<2> The method as described in <1>, wherein the amount of the tau protein accumulated in the brain of the subject is represented by an SUVR value or an AD tau score in a tau PET image.
<3> The method as described in <1> or <2>, wherein the brain is at least one site selected from the group consisting of hippocampus, entorhinal cortex, and neocortex.
<4> The method as described in any one of <1> through <3>, wherein a blocking agent or a blood serum component which is derived from a mammal is added to a buffer solution used to dilute the biological sample.
<5> The method as described in any one of <1> through <4>, wherein in the detecting step, the measurement sample is brought into contact with a substrate having a plurality of microwells, the capture bead and the non-capture bead in the measurement sample are respectively placed in the plurality of microwells, the signal derived from the immune complex on the capture bead placed in any of the plurality of microwells is detected, and each of the plurality of microwells has a volume such that merely a single capture bead or a single non-capture bead can be held in the each of the plurality of microwells.
<6> The method as described in any one of <1> through <5>, wherein, in the detecting step, detection of the signal is carried out by capturing an image of the signal derived from the immune complex.
<7> The method as described in any one of <1> through <6>, wherein the biological sample is a blood sample.
<8> The method as described in <7>, wherein the blood sample is blood serum or blood plasma.
<9> The method as described in any one of <1> through <8>, wherein the immune complex is a complex of the capture antibody, the phosphorylated tau protein in the biological sample, and the detection antibody, and the capture antibody binds to the capture bead.
<10> The method as described in any one of <1> through <9>, wherein the immune complex is formed by mixing the capture antibody which binds to the capture bead, the biological sample which contains the phosphorylated tau protein, and the detection antibody in the presence of the non-capture bead.
<11> A method for obtaining information which assists in determining whether or not a subject is at Braak stage I/II or Braak stage III or higher, based on an amount of a tau protein accumulated in a brain which amount has been estimated by the method described in any one of <1> through <10>.
<12> A method for obtaining information which assists in determining evaluation of a degree of progression of a tauopathy in a subject, based on an amount of a tau protein accumulated in a brain which amount has been estimated by the method described in any one of <1> through <10>.
<13> A reagent or reagent kit used in the method described in any one of <1> through <11>, the reagent or reagent kit including the capture bead and the non-capture bead.
<14> A device which estimates an amount of a tau protein accumulated in a brain of a subject, the device including a processing section, wherein regarding a measurement sample which has been prepared by forming, on a capture bead, an immune complex of a phosphorylated tau protein in a biological sample collected from a subject, a detection antibody, and a capture antibody in the presence of a non-capture bead and which contains the non-capture bead and the capture bead to which the immune complex binds, the processing section obtains a signal derived from the immune complex, the processing section estimates, based on intensity of the signal, an amount of a tau protein accumulated in a brain of the subject which amount is quantified by tau PET, the non-capture bead does not bind to the immune complex, one of the detection antibody and the capture antibody is an antibody which recognizes an amino acid sequence from position 151 to position 244 in an amino acid sequence represented by SEQ ID NO. 1, the other of the detection antibody and the capture antibody is an antibody which recognizes a phosphorylated epitope including a phosphorylated amino acid residue in the amino acid sequence represented by SEQ ID NO. 1, and the intensity of the signal does not correlate with an amount of amyloid accumulated in the brain of the subject which amount is quantified by amyloid PET.
<15> A device which obtains information for evaluating a degree of progression of a tauopathy in a subject, based on intensity of a signal derived from a phosphorylated tau protein in a biological sample collected from the subject, the device including a processing section, wherein regarding a measurement sample which has been prepared by forming, on a capture bead, an immune complex of a phosphorylated tau protein in a biological sample collected from a subject, a detection antibody, and a capture antibody in the presence of a non-capture bead and which contains the non-capture bead and the capture bead to which the immune complex binds, the processing section obtains a signal derived from the immune complex, the processing section estimates, based on intensity of the signal, an amount of a tau protein accumulated in a brain of the subject which amount is quantified by tau PET, the processing section obtains information for evaluating a degree of progression of a tauopathy in the subject, based on the amount of the tau protein accumulated in the brain of the subject which amount has been estimated, the non-capture bead does not bind to the immune complex, one of the detection antibody and the capture antibody is an antibody which recognizes an amino acid sequence from position 151 to position 244 in an amino acid sequence represented by SEQ ID NO. 1, the other of the detection antibody and the capture antibody is an antibody which recognizes a phosphorylated epitope including a phosphorylated amino acid residue in the amino acid sequence represented by SEQ ID NO. 1, and the intensity of the signal does not correlate with an amount of amyloid accumulated in the brain of the subject which amount is quantified by amyloid PET.
<16> A computer program for estimating an amount of a tau protein accumulated in a brain of a subject, the computer program causing a computer to execute: a step of, regarding a measurement sample which has been prepared by forming, on a capture bead, an immune complex of a phosphorylated tau protein in a biological sample, a detection antibody, and a capture antibody in the presence of a non-capture bead and which contains the non-capture bead and the capture bead to which the immune complex binds, obtaining a signal derived from the immune complex, wherein the biological sample is collected from a subject, the non-capture bead does not bind to the immune complex, one of the detection antibody and the capture antibody is an antibody which recognizes an amino acid sequence from position 151 to position 244 in an amino acid sequence represented by SEQ ID NO. 1, and the other of the detection antibody and the capture antibody is an antibody which recognizes a phosphorylated epitope including a phosphorylated amino acid residue in the amino acid sequence represented by SEQ ID NO. 1; and a step of estimating, based on intensity of the signal, an amount of a tau protein accumulated in a brain of the subject which amount is quantified by tau PET, wherein the intensity of the signal does not correlate with an amount of amyloid accumulated in the brain of the subject which amount is quantified by amyloid PET.
<17> A computer program for obtaining information for evaluating a degree of progression of a tauopathy, the computer program causing a computer to execute: a step of, regarding a measurement sample which has been prepared by forming, on a capture bead, an immune complex of a phosphorylated tau protein in a biological sample, a detection antibody, and a capture antibody in the presence of a non-capture bead and which contains the non-capture bead and the capture bead to which the immune complex binds, obtaining a signal derived from the immune complex, wherein the biological sample is collected from a subject, the non-capture bead does not bind to the immune complex, one of the detection antibody and the capture antibody is an antibody which recognizes an amino acid sequence from position 151 to position 244 in an amino acid sequence represented by SEQ ID NO. 1, and the other of the detection antibody and the capture antibody is an antibody which recognizes a phosphorylated epitope including a phosphorylated amino acid residue in the amino acid sequence represented by SEQ ID NO. 1; a step of estimating, based on intensity of the signal, an amount of a tau protein accumulated in a brain of the subject which amount is quantified by tau PET; and a step of obtaining information for evaluating a degree of progression of a tauopathy in the subject, from whom/which the biological sample has been collected, based on the amount of the tau protein accumulated in the brain of the subject which amount has been estimated, wherein the intensity of the signal does not correlate with an amount of amyloid accumulated in the brain of the subject which amount is quantified by amyloid PET.
<18> A method for diagnosing a tauopathy, the method including a diagnosing step of diagnosing whether or not a subject is affected by a tauopathy, based on an amount of a tau protein accumulated in a brain which amount has been estimated by the method described in any one of <1> through <10>.
<19> A method for assisting in diagnosing a patient, the method including a step of obtaining data that suggests whether or not a biological sample is a biological sample derived from a patient affected by a tauopathy, based on an amount of a tau protein accumulated in a brain which amount has been estimated by the method described in any one of <1> through <10>.
<20> A method for diagnosing a Braak stage, the method including a diagnosing step of diagnosing whether or not a subject is at Braak stage I/II or Braak stage III or higher, based on an amount of a tau protein accumulated in a brain which amount has been estimated by the method described in any one of <1> through <10>.
<21> A method for assisting in diagnosing a patient, the method including a step of obtaining data that suggests whether or not a biological sample is a biological sample derived from a patient at Braak stage III or higher, based on an amount of a tau protein accumulated in a brain which amount has been estimated by the method described in any one of <1> through <10>.
<22> A device which obtains information on whether or not a subject is at Braak stage I/II or Braak stage III or higher, based on intensity of a signal derived from a phosphorylated tau protein in a biological sample collected from the subject, the device including a processing section, wherein regarding a measurement sample which has been prepared by forming, on a capture bead, an immune complex of a phosphorylated tau protein in a biological sample collected from a subject, a detection antibody, and a capture antibody in the presence of a non-capture bead and which contains the non-capture bead and the capture bead to which the immune complex binds, the processing section obtains a signal derived from the immune complex, the processing section estimates, based on intensity of the signal, an amount of a tau protein accumulated in a brain of the subject which amount is quantified by tau PET, the processing section obtains information on whether or not the subject is at Braak stage I/II or Braak stage III or higher, based on the amount of the tau protein accumulated in the brain of the subject which amount has been estimated, the non-capture bead does not bind to the immune complex, one of the detection antibody and the capture antibody is an antibody which recognizes an amino acid sequence from position 151 to position 244 in an amino acid sequence represented by SEQ ID NO. 1, the other of the detection antibody and the capture antibody is an antibody which recognizes a phosphorylated epitope including a phosphorylated amino acid residue in the amino acid sequence represented by SEQ ID NO. 1, and the intensity of the signal does not correlate with an amount of amyloid accumulated in the brain of the subject which amount is quantified by amyloid PET.
<23> A computer program for obtaining information on whether or not a subject is at Braak stage I/II or Braak stage III or higher, the computer program causing a computer to execute: a step of, regarding a measurement sample which has been prepared by forming, on a capture bead, an immune complex of a phosphorylated tau protein in a biological sample, a detection antibody, and a capture antibody in the presence of a non-capture bead and which contains the non-capture bead and the capture bead to which the immune complex binds, obtaining a signal derived from the immune complex, wherein the biological sample is collected from a subject, the non-capture bead does not bind to the immune complex, one of the detection antibody and the capture antibody is an antibody which recognizes an amino acid sequence from position 151 to position 244 in an amino acid sequence represented by SEQ ID NO. 1, and the other of the detection antibody and the capture antibody is an antibody which recognizes a phosphorylated epitope including a phosphorylated amino acid residue in the amino acid sequence represented by SEQ ID NO. 1; a step of estimating, based on intensity of the signal, an amount of a tau protein accumulated in a brain of the subject which amount is quantified by tau PET; and a step of obtaining information on whether or not the subject, from whom/which the biological sample has been collected, is at Braak stage I/II or Braak stage III or higher, based on the amount of the tau protein accumulated in the brain of the subject which amount has been estimated, wherein the intensity of the signal does not correlate with an amount of amyloid accumulated in the brain of the subject which amount is quantified by amyloid PET.

Embodiments of the present invention further include aspects described below.
<24> A method for assisting in determination for estimating tau pathology in a brain of a subject, the method including: a preparing step of forming, on a capture bead, an immune complex of a phosphorylated tau protein in a biological sample collected from a subject, a capture antibody, and a detection antibody in the presence of a non-capture bead to prepare a measurement sample containing the non-capture bead and the capture bead to which the immune complex binds; a detecting step of detecting a signal derived from the immune complex in the measurement sample; and an estimating step of estimating, based on intensity of the signal, an amount of a tau protein accumulated in a brain of the subject which amount is quantified by tau PET, wherein the non-capture bead does not bind to the immune complex, one of the detection antibody and the capture antibody is an antibody which recognizes an amino acid sequence from position 151 to position 244 in an amino acid sequence represented by SEQ ID NO. 1, and the other of the detection antibody and the capture antibody is an antibody which recognizes a phosphorylated epitope including a phosphorylated amino acid residue in the amino acid sequence represented by SEQ ID NO. 1.
<25> The method as described in <24>, wherein the amount of the tau protein accumulated in the brain of the subject is represented by an SUVR value or an AD tau score in a tau PET image.
<26> The method as described in <24> or <25>, wherein the brain is at least one site selected from the group consisting of hippocampus, entorhinal cortex, and neocortex.
<27> The method as described in any one of <24> through <26>, wherein a blocking agent or a blood serum component which is derived from a mammal is added to a buffer solution used to dilute the biological sample.
<28> The method as described in any one of <24> through <27>, wherein in the detecting step, the measurement sample is brought into contact with a substrate having a plurality of microwells, the capture bead and the non-capture bead in the measurement sample are respectively placed in the plurality of microwells, the signal derived from the immune complex on the capture bead placed in any of the plurality of microwells is detected, and each of the plurality of microwells has a volume such that merely a single capture bead or a single non-capture bead can be held in the each of the plurality of microwells.
<29> The method as described in any one of <24> through <28>, wherein in the detecting step, detection of the signal is carried out by capturing an image of the signal derived from the immune complex.
<30> The method as described in any one of <24> through <29>, wherein the biological sample is a blood sample.
<31> The method as described in <30>, wherein the blood sample is blood serum or blood plasma.
<32> The method as described in any one of <24> through <31>, wherein the immune complex is a complex of the capture antibody, the phosphorylated tau protein in the biological sample, and the detection antibody, and the capture antibody binds to the capture bead.
<33> The method as described in any one of <24> through <32>, wherein the immune complex is formed by mixing the capture antibody which binds to the capture bead, the biological sample which contains the phosphorylated tau protein, and the detection antibody in the presence of the non-capture bead.
<34> A method for obtaining information which assists in determining whether or not a subject is at Braak stage I/II or Braak stage III or higher, based on an amount of a tau protein accumulated in a brain which amount has been estimated by the method described in any one of <24> through <33>.
<35> A method for obtaining information which assists in evaluating a degree of progression of a tauopathy in a subject, based on an amount of a tau protein accumulated in a brain which amount has been estimated by the method described in any one of <24> through <33>.
<36> A reagent or reagent kit used in the method described in any one of <24> through <33>, the reagent or reagent kit including the capture bead and the non-capture bead.
<37> A device which obtains information for estimating tau pathology in a brain of a subject, the device including a processing section, wherein regarding a measurement sample which has been prepared by forming, on a capture bead, an immune complex of a phosphorylated tau protein in a biological sample collected from a subject, a detection antibody, and a capture antibody in the presence of a non-capture bead and which contains the non-capture bead and the capture bead to which the immune complex binds, the processing section obtains a signal derived from the immune complex, the processing section estimates, based on intensity of the signal, an amount of a tau protein accumulated in a brain of the subject which amount is quantified by tau PET, the non-capture bead does not bind to the immune complex, one of the detection antibody and the capture antibody is an antibody which recognizes an amino acid sequence from position 151 to position 244 in an amino acid sequence represented by SEQ ID NO. 1, and the other of the detection antibody and the capture antibody is an antibody which recognizes a phosphorylated epitope including a phosphorylated amino acid residue in the amino acid sequence represented by SEQ ID NO. 1.
<38> A device which obtains information for evaluating a degree of progression of a tauopathy in a subject, based on intensity of a signal derived from a phosphorylated tau protein in a biological sample collected from the subject, the device including a processing section, wherein regarding a measurement sample which has been prepared by forming, on a capture bead, an immune complex of a phosphorylated tau protein in a biological sample collected from a subject, a detection antibody, and a capture antibody in the presence of a non-capture bead and which contains the non-capture bead and the capture bead to which the immune complex binds, the processing section obtains a signal derived from the immune complex, the processing section estimates, based on intensity of the signal, an amount of a tau protein accumulated in a brain of the subject which amount is quantified by tau PET, the processing section obtains information for evaluating a degree of progression of a tauopathy in the subject, based on the amount of the tau protein accumulated in the brain of the subject which amount has been estimated, the non-capture bead does not bind to the immune complex, one of the detection antibody and the capture antibody is an antibody which recognizes an amino acid sequence from position 151 to position 244 in an amino acid sequence represented by SEQ ID NO. 1, and the other of the detection antibody and the capture antibody is an antibody which recognizes a phosphorylated epitope including a phosphorylated amino acid residue in the amino acid sequence represented by SEQ ID NO. 1.
<39> A device which obtains information on whether or not a subject is at Braak stage I/II or Braak stage III or higher, based on intensity of a signal derived from a phosphorylated tau protein in a biological sample collected from the subject, the device including a processing section, wherein regarding a measurement sample which has been prepared by forming, on a capture bead, an immune complex of a phosphorylated tau protein in a biological sample collected from a subject, a detection antibody, and a capture antibody in the presence of a non-capture bead and which contains the non-capture bead and the capture bead to which the immune complex binds, the processing section obtains a signal derived from the immune complex, the processing section estimates, based on intensity of the signal, an amount of a tau protein accumulated in a brain of the subject which amount is quantified by tau PET, the processing section obtains information on whether or not the subject is at Braak stage I/II or Braak stage III or higher, based on the amount of the tau protein accumulated in the brain of the subject which amount has been estimated, the non-capture bead does not bind to the immune complex, one of the detection antibody and the capture antibody is an antibody which recognizes an amino acid sequence from position 151 to position 244 in an amino acid sequence represented by SEQ ID NO. 1, and the other of the detection antibody and the capture antibody is an antibody which recognizes a phosphorylated epitope including a phosphorylated amino acid residue in the amino acid sequence represented by SEQ ID NO. 1.
<40> A computer program for obtaining information for estimating tau pathology in a brain of a subject, the computer program causing a computer to execute: a step of, regarding a measurement sample which has been prepared by forming, on a capture bead, an immune complex of a phosphorylated tau protein in a biological sample, a detection antibody, and a capture antibody in the presence of a non-capture bead and which contains the non-capture bead and the capture bead to which the immune complex binds, obtaining a signal derived from the immune complex, wherein the biological sample is collected from a subject, the non-capture bead does not bind to the immune complex, one of the detection antibody and the capture antibody is an antibody which recognizes an amino acid sequence from position 151 to position 244 in an amino acid sequence represented by SEQ ID NO. 1, and the other of the detection antibody and the capture antibody is an antibody which recognizes a phosphorylated epitope including a phosphorylated amino acid residue in the amino acid sequence represented by SEQ ID NO. 1; and a step of estimating, based on intensity of the signal, an amount of a tau protein accumulated in a brain of the subject which amount is quantified by tau PET.
<41> A computer program for obtaining information for evaluating a degree of progression of a tauopathy, the computer program causing a computer to execute: a step of, regarding a measurement sample which has been prepared by forming, on a capture bead, an immune complex of a phosphorylated tau protein in a biological sample, a detection antibody, and a capture antibody in the presence of a non-capture bead and which contains the non-capture bead and the capture bead to which the immune complex binds, obtaining a signal derived from the immune complex, wherein the biological sample is collected from a subject, the non-capture bead does not bind to the immune complex, one of the detection antibody and the capture antibody is an antibody which recognizes an amino acid sequence from position 151 to position 244 in an amino acid sequence represented by SEQ ID NO. 1, and the other of the detection antibody and the capture antibody is an antibody which recognizes a phosphorylated epitope including a phosphorylated amino acid residue in the amino acid sequence represented by SEQ ID NO. 1; a step of estimating, based on intensity of the signal, an amount of a tau protein accumulated in a brain of the subject which amount is quantified by tau PET; and a step of obtaining information for evaluating a degree of progression of a tauopathy in the subject, from whom/which the biological sample has been collected, based on the amount of the tau protein accumulated in the brain of the subject which amount has been estimated.
<42> A computer program for obtaining information on whether or not a subject is at Braak stage I/II or Braak stage III or higher, the computer program causing a computer to execute: a step of, regarding a measurement sample which has been prepared by forming, on a capture bead, an immune complex of a phosphorylated tau protein in a biological sample, a detection antibody, and a capture antibody in the presence of a non-capture bead and which contains the non-capture bead and the capture bead to which the immune complex binds, obtaining a signal derived from the immune complex, wherein the biological sample is collected from a subject, the non-capture bead does not bind to the immune complex, one of the detection antibody and the capture antibody is an antibody which recognizes an amino acid sequence from position 151 to position 244 in an amino acid sequence represented by SEQ ID NO. 1, and the other of the detection antibody and the capture antibody is an antibody which recognizes a phosphorylated epitope including a phosphorylated amino acid residue in the amino acid sequence represented by SEQ ID NO. 1; a step of estimating, based on intensity of the signal, an amount of a tau protein accumulated in a brain of the subject which amount is quantified by tau PET; and a step of obtaining information on whether or not the subject, from whom/which the biological sample has been collected, is at Braak stage I/II or Braak stage III or higher, based on the amount of the tau protein accumulated in the brain of the subject which amount has been estimated.

An example is provided below to describe the embodiments of the present invention in more detail. Obviously, the present invention is not limited to the example below, and it is needless to say that various aspects are possible for details. Furthermore, the present invention is not limited to the above-described embodiments, and various modifications can be made within the scope of the claims. The present invention encompasses, in its technical scope, an embodiment obtained by appropriately combining technical means disclosed in differing embodiments. Further, all of the documents listed in the present specification are incorporated herein by reference.

### EXAMPLES

### [Materials]

Capture beads were prepared with the use of beads of Simoa (trademark) Homebrew Assay Development kit available from Quanterix Corporation in accordance with the protocol attached thereto. As non-capture beads, Dye-Encoded Helper Beads available from Quanterix Corporation were used. As a sample buffer, a buffer to which BSA was added was used. As a capture antibody, an anti-human tau protein antibody (HT7, Thermo Fisher Scientific Inc.) was used. HT7 is an antibody which recognizes a peptide from position 159 to position 163 in the amino acid sequence of SEQ ID NO. 1. As a detection antibody, an anti-phosphorylated tau protein antibody (AT270, Thermo Fisher Scientific Inc.) was used. Biotinylation of the detection antibody was carried out in accordance with the protocol of the above kit. AT270 is an antibody which recognizes the phosphorylation of tyrosine at position 181 in SEQ ID NO. 1. Hereinafter, a method for measuring a phosphorylated tau protein with the use of the combination of the above capture antibody and the above detection antibody may be abbreviated as “measurement method of the example”.

As a target protein, a phosphorylated tau protein (p-Tau181) was used. p-Tau181 is a tau protein in which tyrosine at position 181 in SEQ ID NO. 1 is phosphorylated. In order to create a standard curve, a human phosphorylated tau standard peptide (p-Tau181) was used as a standard peptide. The target protein or the standard peptide was diluted with the use of Sample diluent of Simoa (trademark) Homebrew Assay Development kit. Then, measurement was carried out with the use of Simoa (trademark) HD-1 Analyzer in accordance with the protocol provided by the manufacturer.

FIG. 3 schematically shows the phosphorylated tau protein and the antibody set which was used in the measurement method of the example and a measurement method of a comparative example. An antibody which recognizes a middle part of the tau protein in FIG. 3 is the capture antibody which was used in the measurement method of the example. An antibody which recognizes p-Tau181 is the detection antibody which was used in the measurement method of the example. Tau12 is an antibody which recognizes a peptide from position 9 to position 18 in the amino acid sequence of SEQ ID NO. 1. A method for measuring the phosphorylated tau protein with the use of the combination of the capture antibody (AT270) and the detection antibody (Tau12) may be abbreviated as “measurement method of the comparative example”. A fragment of the phosphorylated tau protein which is measured by the measurement method of the example may be referred to as mid-p-tau181. A fragment of the phosphorylated tau protein which is measured by the measurement method of the comparative example may be referred to as N-p-tau181.

### [Evaluation Example 1] Measurement of a Phosphorylated Tau Protein

As samples, used were blood from 48 patients with Alzheimer's disease in whom accumulation in tau pathology was obvious in the cerebrum by tau PET with the use of [¹⁸F]PM-PBB3 as a tau PET ligand, 76 patients with cranial nerve diseases other than Alzheimer's disease, and 40 normal controls who had no accumulation of tau and who were also cognitively normal. In all of these cases, the accumulation of amyloid in the cerebrum had also been examined by amyloid PET with the use of [¹¹C]PiB as an amyloid PET ligand.

The measurement was carried out with the use of Simoa (trademark) Enzyme and Substrate kit, Simoa (trademark) Disk kit, Simoa (trademark) Sealing Oil, System buffer 1, System buffer 2, and Simoa (trademark) HD-1 Analyzer in accordance with the protocol provided by the manufacturer. In the measurement, each measurement sample and a necessary reagent including the capture beads, the biotin-labeled antibody, and the non-capture beads were set in Simoa (trademark) HD-1 Analyzer. Then, all measuring steps were carried out with Simoa (trademark) HD-1 Analyzer.

In Evaluation Example 1, preparation was carried out such that the ratio of the number of the non-capture beads to the number of the capture beads became 3:1 to 9:1, in both the measurement methods of the example and the comparative example. Further, the preparation was carried out such that the concentration of the entire beads (the capture beads and the non-capture beads) became 400,000 to 500,000 beads with respect to 90 μL of each measurement sample.

The detailed measuring steps are as follows. First, the standard protein in blood and the capture antibody which was bound to each of the capture beads were bound to each other in the presence of the non-capture beads. Next, the biotinylated detection antibody was further bound to the standard protein which was bound to the capture antibody, so that an immune complex of the capture antibody, the standard protein, and the biotinylated detection antibody was formed on each of the capture beads.

After formation of the immune complex, the capture beads and β-galactosidase-bound streptavidin were reacted in the presence of the non-capture beads. Then, the capture beads were reacted with a substrate (RGP: resorufin β-D-galactopyranoside) of β-galactosidase in the presence of the non-capture beads. The non-capture beads and the capture beads which had completed reacting with the RGP were applied to Simoa (trademark) Disk. Thereafter, the applied beads were stored in respective wells in an array in which a large number of wells of a femtoliter (fL) unit were arranged. An image of each of the beads stored in the respective wells was captured with Simoa (trademark) HD-1 Analyzer, and a fluorescence intensity (AEB) was measured. The target protein was quantified by measuring the number of beads which each had a high fluorescence intensity.

### [Evaluation Example 2] Investigation of the Correlation Between the Measured Value of p-Tau181 in the Blood and the Amount of an Accumulated Tau Protein which had been Quantified by Tau PET

It was investigated whether or not the measured value of p-Tau181 in the blood (hereinafter may be referred to as “blood p-Tau181”) which had been measured in Evaluation Example 1 correlated with the amount of an accumulated tau protein which had been quantified by tau PET.

In obtaining a tau PET image, [¹⁸F]PM-PBB3 was used as a tau PET ligand.

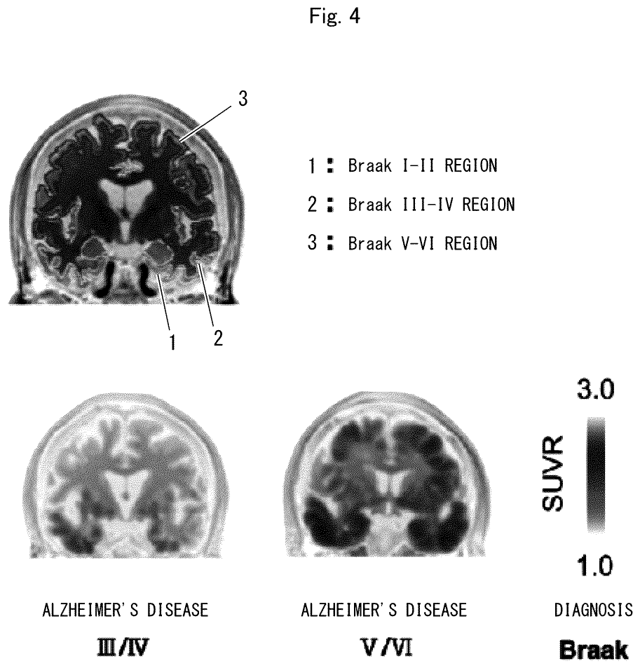

In determining the amount of the accumulated tau protein by tau PET, the “Braak stages”, which are international stage classifications that reflect the pathological spread of the accumulation of a tau protein in Alzheimer's disease, were used. The amount of the accumulated tau protein by tau PET was obtained by calculating, from the tau PET image, a standard uptake value ratio (SUVR) value, which represents the amount of a tau protein accumulated in a brain site(s) corresponding to Braak stage I/II, III/IV, or V/VI (FIG. 4). In calculating the SUVR, the cerebellar cortex was used as a non-specific accumulation exhibiting brain site. In obtaining an amyloid PET image, [¹¹C]PiB was used as an amyloid PET ligand.

At Braak stage I/II, the accumulation of a tau protein is observed in the entorhinal cortex region of the cerebrum. At Braak stage III/IV, the accumulation of the tau protein is also observed in the limbic system region of the cerebrum, such as the hippocampus, and in the site of the inferior temporal lobe. At Braak stage V/VI, the accumulation of the tau protein which has spread in the neocortical region of the cerebrum is observed. In the tau PET image obtained from an actual patient, brain sites corresponding to the above Braak stage I/II, III/IV, V/VI regions were identified as in the upper left image in FIG. 4, and the SUVR value was calculated for each of the brain sites.

The accumulation of tau in the site corresponding to Braak stage I/II is also observed in individuals with normal aging. However, it is known that the accumulation of the tau protein in patients with Alzheimer's disease reaches Braak stage III/IV or higher, as shown in the lower images in FIG. 4. Namely, in patients with Alzheimer's disease, the accumulation of tau in the brain is not limited to the Braak stage I/II region, but expands to the Braak stage III/IV region at a stage at which cognitive impairment begins. In patients with Alzheimer's disease, the accumulation of tau further spreads to the Braak stage V/VI region at a stage at which cognitive impairment has progressed.

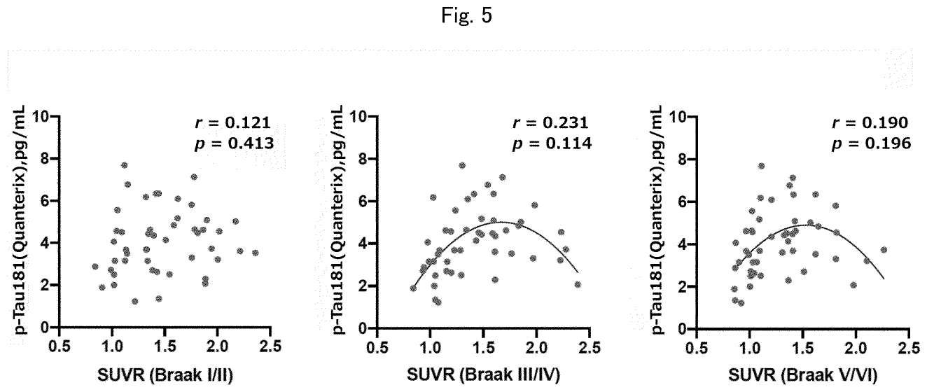

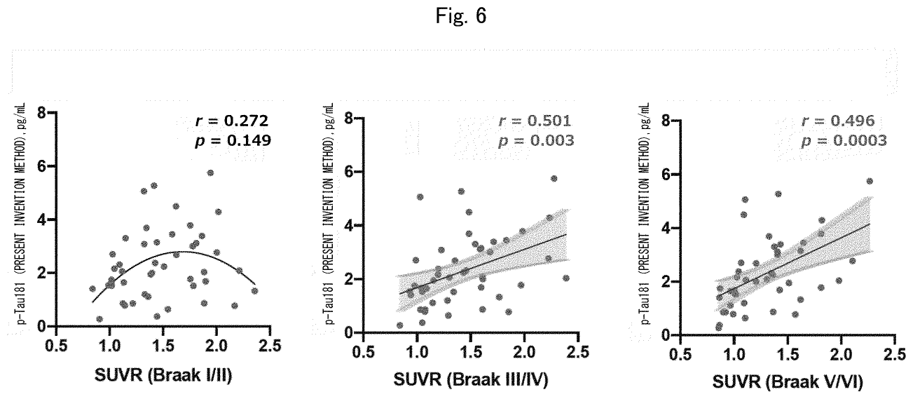

FIG. 5 shows graphs in each of which the concentrations of blood p-Tau181 of 48 patients with Alzheimer's disease that were measured by the measurement method of the comparative example are plotted on the vertical axis and SUVR values in the brain site(s) corresponding to each stage (stage I/II, III/IV, or V/VI) in the “Braak stages” are plotted on the horizontal axis. FIG. 6 shows graphs in each of which the concentrations of blood p-Tau181 of 48 patients with Alzheimer's disease that were measured by the measurement method of the example are plotted on the vertical axis and the SUVR values at each stage in the “Braak stages” are plotted on the horizontal axis. In FIGS. 5 and 6, it is investigated whether or not the amounts (SUVR values) of the tau protein accumulated in the brain sites corresponding to Braak stage I/II, III/IV, and V/VI correlate with the concentrations of blood p-Tau181 which were measured by the measurement methods of the example and the comparative example. These investigations were carried out by Pearson's product-moment correlation coefficient. In the absence of a significant linear correlation, a non-linear regression analysis was carried out. All of these statistical analyses were carried out by GraphPad Prism version 9 (GraphPad Software).

As shown in FIG. 5, the concentrations of blood p-Tau181 which had been measured by the measurement method of the comparative example did not correlate with the amounts of the tau protein accumulated in the brain site region corresponding to each Braak stage, the amounts having been quantified by tau PET. In the brain site regions corresponding to Braak stage III/IV (middle graph in FIG. 5) and V/VI (right graph in FIG. 5), there was a tendency for the concentration of blood p-Tau181 to decrease as the amount of the accumulated tau protein by tau PET increased. Namely, the concentrations of blood p-Tau181 which had been quantified by the measurement method of the comparison example did not reflect the amounts of the tau protein accumulated in the brain which had been quantified by tau PET.

In contrast, as shown in FIG. 6, a significant positive correlation was observed between the concentrations of blood p-Tau181 which had been measured by the measurement method of the example and the amounts of the abnormal tau protein accumulated in the brain site regions corresponding to Braak stages III/IV and V/VI, at which the accumulation of a tau protein is observed in patients with Alzheimer's disease. This result showed that the concentration of blood p-Tau181 which had been measured by the measurement method of the example accurately reflected the amount of the tau protein accumulated in the brain of the actual patient.

### [Evaluation Example 3] Discrimination Between Braak Stage I/II and Braak Stage III or Higher by the Concentration of Blood p-Tau181

In the case of normal aging, the accumulation of a tau protein remains limited to Braak stage I/II, whereas, in the brain of a patient with Alzheimer's disease, the accumulation advances to Braak stage III or higher. The concentrations of blood p-Tau181 which had been measured in Evaluation Example 1 were used to investigate whether or not it was possible to discriminate between the localized accumulation of tau due to normal aging (up to Braak stage I/II) and the spread accumulation of tau which corresponded to Alzheimer's disease (Braak stage III or higher).

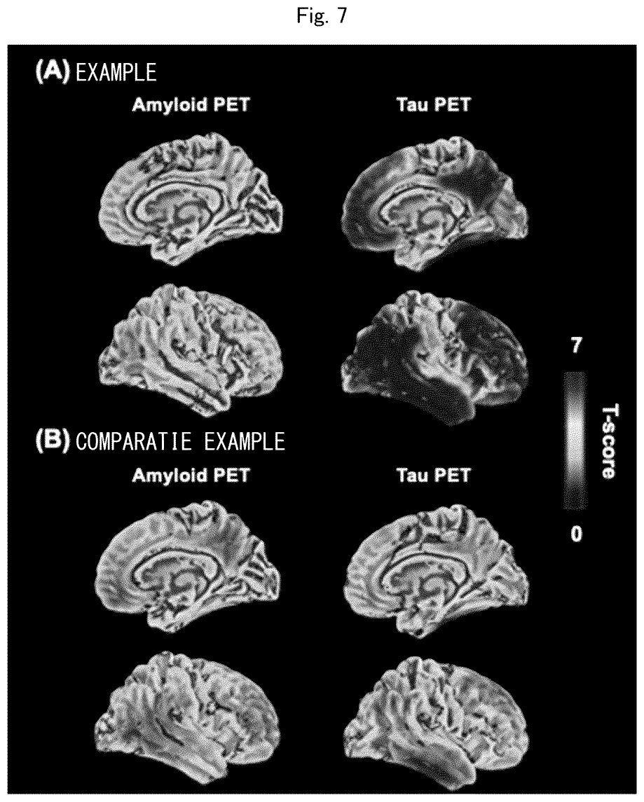

In a total of 88 subjects including 48 patients with Alzheimer's disease and 40 normal controls who had no accumulation of the tau protein and who were also cognitively normal, investigations were carried out on at which sites in the cerebrum and how strongly the concentrations of blood p-Tau181 which had been measured by the measurement methods of the example and the comparative example correlated with the amounts of amyloid and tau deposited in the cerebrum which had respectively been quantified by amyloid PET and tau PET. A result is shown in FIG. 7.

FIG. 7 shows, in a total of 88 subjects including the patients with Alzheimer's disease and the normal controls, brain sites in which the concentrations of blood p-Tau181 which had been measured by the measurement methods of the example ((A) in FIG. 7) and the comparative example ((B) in FIG. 7) correlated with the amounts of amyloid and tau deposited in the cerebrum which had respectively been quantified by amyloid PET and tau PET. In FIG. 7, the strength of the correlation with the amounts of deposited amyloid and tau is indicated by color intensity. The concentrations of blood p-Tau181 which had been measured by the measurement method of the example ((A) in FIG. 7) clearly correlated more strongly with the accumulation of the tau protein than with that of amyloid. Statistically, only the accumulation of the tau protein contributed to the concentrations of p-Tau in the example. In contrast, the concentrations of blood p-Tau181 which had been measured by the measurement method of the comparative example ((B) in FIG. 7) correlated more strongly with the accumulation of amyloid. Statistically, both the accumulation of amyloid and the accumulation of the tau protein contributed to the concentrations of p-Tau in the comparative example. From this result, it was found that, unlike the comparative example, the concentrations of blood p-Tau181 which had been measured by the measurement method of the example accurately reflected the accumulation of the tau protein in the cerebrum which had been quantified by tau PET, without being affected by amyloid.

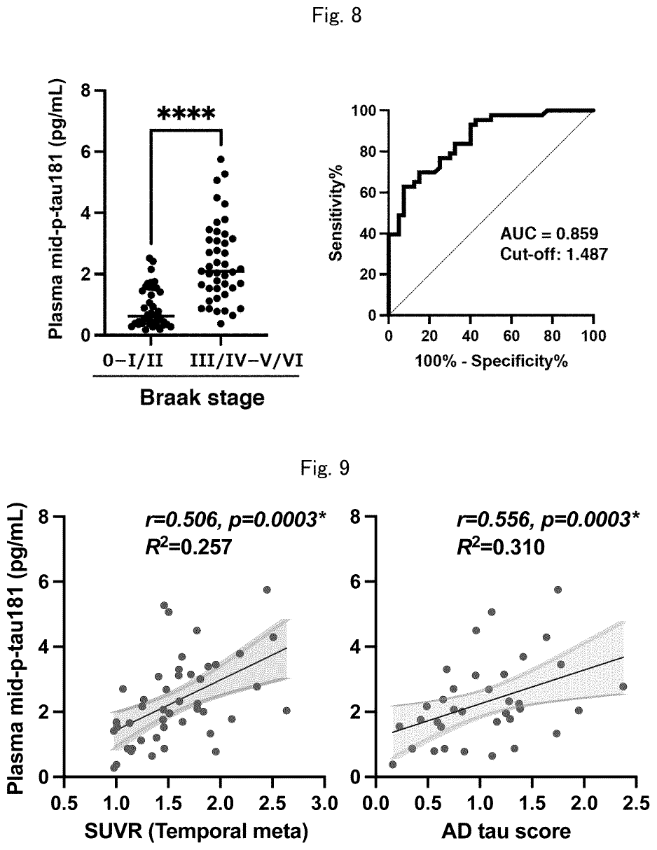

The left graph in FIG. 8 is a graph showing the measured values (concentrations, pg/mL) of blood p-Tau181 of the subjects who had been determined, as a result of tau PET, to be at Braak Stage I/II or Braak Stage III or higher (Braak Stage III/IV or Braak Stage V/VI). The right graph in FIG. 8 is a graph in which, regarding the concentrations of blood p-Tau181, the performance of discriminating between Braak stage I/II and Braak stage III or higher was evaluated by ROC analysis.

As shown in FIG. 8, based on the concentrations of blood p-Tau181 which had been measured by the measurement method of the example, it was possible to discriminate between the accumulation of tau in the human brain at up to Braak stage I/II and the accumulation of tau in the human brain at Braak stage III or higher with high accuracy (AUC of 0.859 in the ROC analysis). From this result, it was found that, based on the concentrations of blood p-Tau181 which had been measured by the measurement method of the example, it was possible to accurately diagnose the localized accumulation of tau due to normal aging (up to Braak I/II) and the advanced accumulation of tau which corresponded to Alzheimer's disease (Braak III or higher), with high consistency with tau PET.

In the present evaluation example, the HT7 antibody was used as an antibody which recognizes a fragment in the middle part of the tau protein (amino acid sequence from position 151 to position 244 in SEQ ID NO. 1). It is evident to a person skilled in the art that the concentration of a blood phosphorylated tau protein which has been measured with the use of an antibody which recognizes a fragment that is in the middle part of the tau protein and that is an epitope differing from that of the HT7 antibody also accurately reflects the amount of the tau protein accumulated in the brain of an actual patient.

In the present evaluation example, the antibody which recognizes phosphorylation of threonine at position 181 in SEQ ID NO. 1 was used as the detection antibody. It is evident to a person skilled in the art that the concentration of the blood phosphorylated tau protein which has been measured with the use of an antibody which recognizes a phosphorylated amino acid differing from the phosphorylated threonine also accurately reflects the amount of the tau protein accumulated in the brain of an actual patient.

### [Evaluation Example 4] Investigation of the Detection of a Reaction by the Addition of a Blocking Agent

The effect of the addition of a blocking agent to a sample buffer on the detection of a reaction was investigated with the use of a sample buffer to which a blocking agent including BSA as a main component was added or a sample buffer to which no blocking agent was added. The AEB of a background was measured twice with the use of Simoa (trademark) Enzyme and Substrate kit, Simoa (trademark) Disk kit, Simoa (trademark) Sealing Oil, System buffer 1, System buffer 2, and Simoa (trademark) HD-1 Analyzer in accordance with the protocol provided by the manufacturer.

In a case where the sample buffer to which the blocking agent was added was used, the AEB was 0.00302±0.001 (mean value±S.D.). In a case where the sample buffer to which no blocking agent was added was used, the AEB was 0.00404±0.000, and the background was reduced by approximately 25% by the addition of the blocking agent. From Evaluation Example 3, it was found that the addition of the blocking agent to the sample buffer reduced the background during the detection of an antigen-antibody reaction.

### [Evaluation Example 5] Investigation of the Correlation Between the Measured Value of Blood p-Tau181 and an AD Tau Score

As an index which indicates the amount of an accumulated tau protein that is quantified by tau PET, the AD Tau scores are known. In the present evaluation example, it was investigated whether or not the measured values of blood p-Tau181 also correlated with the AD Tau scores. The AD Tau scores were calculated with reference to Movement Disorders, 2022, Vol. 37, No. 11:2236-2246.

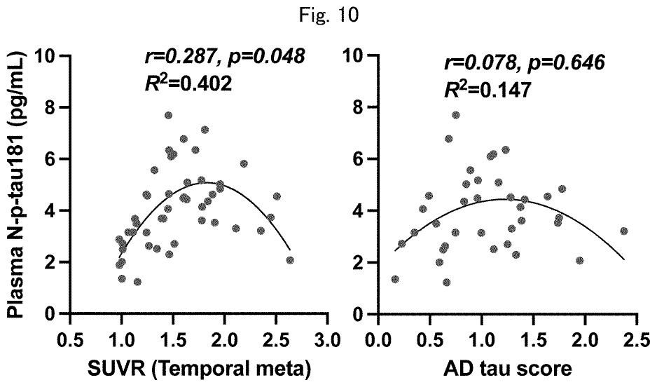

FIG. 9 shows graphs in each of which the concentrations of blood mid-p-tau181 of 48 patients with Alzheimer's disease that were measured by the measurement method of the example are plotted on the vertical axis and the SUVR values (temporal meta ROI) or the AD tau scores are plotted on the horizontal axis. FIG. 10 shows graphs in each of which the concentrations of blood p-Tau181 (N-p-tau181) of 48 patients with Alzheimer's disease that were measured by the measurement method of the comparative example are plotted on the vertical axis and the SUVR values (temporal meta ROI) or the AD tau scores are plotted on the horizontal axis.

As is clear from FIGS. 9 and 10, a significant positive correlation was also observed between the concentrations of blood mid-p-tau181 which had been measured by the measurement method of the example and the AD Tau scores.

### Summary of Example

It is considered that, in the measurement method of the comparative example, a fragment of a phosphorylated tau protein (p-tau) released into the blood in response to amyloid in the brain at a stage before a tau protein accumulates significantly in the brain is detected. Therefore, the concentration of the phosphorylated tau protein which is obtained by the measurement method of the comparative example correlates more strongly with the amount of amyloid accumulated in the brain than the amount of the tau protein accumulated in the brain, and does not purely reflect the amount of the tau protein accumulated in the brain. Therefore, the concentration of the phosphorylated tau protein which is obtained by the measurement method of the comparative example does not correlate with the amount of the accumulated tau protein which is measured by tau PET.

In contrast, it is considered that, in the measurement method of the example, the fragment of the tau protein released into the blood at a stage at which the tau protein accumulates significantly in the brain is detected. Blood mid-p-tau181 measured by the measurement method of the example does not correlate with the amount of amyloid accumulated in the brain which is measured by amyloid PET. Therefore, the concentration of the phosphorylated tau protein which is obtained by the measurement method of the example correlates with the amount of the accumulated tau protein which is measured by tau PET, without being affected by the amount of amyloid accumulated in the brain.

It has been revealed that, in Alzheimer's disease, the accumulation of amyloid in the brain and tau pathology appear before cognitive impairment appears. In the actual cognitively normal elderly, even in a case where the accumulation of amyloid in the brain is positive, cognitive impairment does not develop during the following approximately five years, provided that tau pathology is negative. However, it has been found that, in a case where the accumulation of amyloid in the brain and tau pathology are positive, the risk of developing cognitive impairment within five years increases. By the combination of the antibodies used in the measurement method of the example, a blood p-Tau biomarker which makes it possible to more accurately estimate the amount of a tau protein accumulated in the brain was obtained. The measurement method of the example makes it possible to diagnose or select, among the general elderly, a patient in whom the onset of dementia is imminent. The measurement method of the example also makes it possible to provide early medical intervention to a patient who is at high risk of developing dementia.

An anti-amyloid drug (such as lecanemab (Eisai)) which is a new radical therapeutic agent for Alzheimer's disease and which is expected to be approved in Japan in 2023 can reduce the accumulation of amyloid in the brain, and is therefore effective in a patient in whom only amyloid is accumulated in the brain. On the other hand, the effect of the anti-amyloid drug cannot be fully expected in a patient in whom a pathological cascade starting from the accumulation of tau and tau pathology at Braak stage III or higher, which are induced by amyloid, has already begun. Therefore, as a patient to whom the anti-amyloid drug can be administered, a patient in whom tau pathology has not yet advanced to Braak stage III or higher (remaining at I/II) is suitable. Conventionally, in order to confirm a Braak stage of a patient, there has been only a method of using tau PET, which is expensive and invasive and which can be used in limited facilities. However, by the combination of the antibodies used in the measurement method of the example, a blood p-Tau biomarker which makes it possible to more accurately estimate the amount of a tau protein accumulated in the brain was obtained. The measurement method of the example makes it possible to select, among the general elderly, a patient in whom tau pathology in the brain has not yet advanced to Braak stage III or higher (remaining at I/II). Therefore, the measurement method of the example makes it possible to select a patient for whom the effect of the anti-amyloid drug can be most expected, not only in actual clinical practice but also in a clinical trial of the anti-amyloid drug.

In a case where the accumulation of a tau protein in the brain progresses, it is not possible to stop the progression of Alzheimer's disease pathogenesis, i.e., the induction of nerve cell death by the accumulation of the tau protein, downstream of amyloid, even when an anti-amyloid drug is administered to reduce the amount of amyloid in a target. Therefore, it is necessary to determine the degree of accumulation of the tau protein in the brain before the administration of the anti-amyloid drug. As described above, since a phosphorylated tau protein which is measured by the conventional method for measuring a phosphorylated tau protein includes a phosphorylated tau protein which is released in response to the accumulation of amyloid in the brain, it is not possible to determine the pure amount of a tau protein accumulated in the brain. Therefore, also in the “selection of a suitable patient to whom an anti-amyloid drug should be administered”, the development of a biological sample biomarker which purely reflects the amount of a tau protein accumulated in the brain is important. At a stage at which the number of patients with a disease such as Alzheimer's disease is very high, it is impossible, due to examination capacity and medical economy, to carry out the selection of such a patient after, in addition to amyloid PET imaging, tau PET imaging is also carried out. The measurement method of the example makes it possible to purely determine the amount of a tau protein accumulated in the brain, and makes it possible to select, in a simple manner without the use of amyloid PET or tau PET, a suitable patient to whom an anti-amyloid drug should be administered.

From the above, the measurement method of the example was found to be useful as a method for accurately reflecting the amount of a tau protein accumulated in the brain. The measurement method of the example can be expected to have applications in actual clinical practice for cognitive impairment and in clinical trials for anti-Alzheimer's drugs.

### INDUSTRIAL APPLICABILITY

The method in accordance with an embodiment of the present invention makes it possible to provide information which can be used for a highly sensitive biochemical diagnosis of a tauopathy such as Alzheimer's disease, and can be used, for example, in a pharmaceutical application and the like.

### REFERENCE SIGNS LIST

20 Measuring device
21, 51 Processing section (CPU)
22, 52 Main storage section
23, 53 ROM
24, 54 Auxiliary storage section
25, 55 Input I/F
26, 56 Output I/F
27, 57 Media I/F
28, 58 Communication I/F
29, 59 Bus
30, 60 Input section
31, 61 Output section
32, 62 Storage medium
50 Information input device

1. A method for estimating an amount of a tau protein accumulated in a brain of a subject, the method comprising:
a preparing step of forming, on a capture bead, an immune complex of a phosphorylated tau protein in a biological sample collected from a subject, a capture antibody, and a detection antibody in the presence of a non-capture bead to prepare a measurement sample containing the non-capture bead and the capture bead to which the immune complex binds;
a detecting step of detecting a signal derived from the immune complex in the measurement sample; and
an estimating step of estimating, based on intensity of the signal, an amount of a tau protein accumulated in a brain of the subject which amount is quantified by tau PET, wherein
the non-capture bead does not bind to the immune complex,
one of the detection antibody and the capture antibody is an antibody which recognizes an amino acid sequence from position 151 to position 244 in an amino acid sequence represented by SEQ ID NO. 1,
the other of the detection antibody and the capture antibody is an antibody which recognizes a phosphorylated epitope including a phosphorylated amino acid residue in the amino acid sequence represented by SEQ ID NO. 1, and
the intensity of the signal does not correlate with an amount of amyloid accumulated in the brain of the subject which amount is quantified by amyloid PET.

2. The method as set forth in claim 1, wherein the amount of the tau protein accumulated in the brain of the subject is represented by an SUVR value or an AD tau score in a tau PET image.

3. The method as set forth in claim 1, wherein the brain is at least one site selected from the group consisting of hippocampus, entorhinal cortex, and neocortex.

4. The method as set forth in claim 1, wherein a blocking agent or a blood serum component which is derived from a mammal is added to a buffer solution used to dilute the biological sample.

5. The method as set forth in claim 1, wherein
in the detecting step,
the measurement sample is brought into contact with a substrate having a plurality of microwells,
the capture bead and the non-capture bead in the measurement sample are respectively placed in the plurality of microwells,
the signal derived from the immune complex on the capture bead placed in any of the plurality of microwells is detected, and
each of the plurality of microwells has a volume such that merely a single capture bead or a single non-capture bead can be held in the each of the plurality of microwells.

6. The method as set forth in claim 1, wherein, in the detecting step, detection of the signal is carried out by capturing an image of the signal derived from the immune complex.

7. The method as set forth in claim 1, wherein the biological sample is a blood sample.

8. The method as set forth in claim 7, wherein the blood sample is blood serum or blood plasma.

9. The method as set forth in claim 1, wherein the immune complex is a complex of the capture antibody, the phosphorylated tau protein in the biological sample, and the detection antibody, and the capture antibody binds to the capture bead.

10. The method as set forth in claim 1, wherein the immune complex is formed by mixing the capture antibody which binds to the capture bead, the biological sample which contains the phosphorylated tau protein, and the detection antibody in the presence of the non-capture bead.

11. A method for obtaining information which assists in determining whether or not a subject is at Braak stage I/II or Braak stage III or higher, based on an amount of a tau protein accumulated in a brain which amount has been estimated by the method recited in claim 1.

12. A method for obtaining information which assists in evaluating a degree of progression of a tauopathy in a subject, based on an amount of a tau protein accumulated in a brain which amount has been estimated by the method recited in claim 1.

13. A reagent or reagent kit used in the method recited in claim 1, the reagent or reagent kit comprising the capture bead and the non-capture bead.

14. A device which estimates an amount of a tau protein accumulated in a brain of a subject,
the device comprising a processing section, wherein
regarding a measurement sample which has been prepared by forming, on a capture bead, an immune complex of a phosphorylated tau protein in a biological sample collected from a subject, a detection antibody, and a capture antibody in the presence of a non-capture bead and which contains the non-capture bead and the capture bead to which the immune complex binds, the processing section obtains a signal derived from the immune complex,
the processing section estimates, based on intensity of the signal, an amount of a tau protein accumulated in a brain of the subject which amount is quantified by tau PET,
the non-capture bead does not bind to the immune complex,
one of the detection antibody and the capture antibody is an antibody which recognizes an amino acid sequence from position 151 to position 244 in an amino acid sequence represented by SEQ ID NO. 1,
the other of the detection antibody and the capture antibody is an antibody which recognizes a phosphorylated epitope including a phosphorylated amino acid residue in the amino acid sequence represented by SEQ ID NO. 1, and
the intensity of the signal does not correlate with an amount of amyloid accumulated in the brain of the subject which amount is quantified by amyloid PET.

15. A device which obtains information for evaluating a degree of progression of a tauopathy in a subject, based on intensity of a signal derived from a phosphorylated tau protein in a biological sample collected from the subject,
the device comprising a processing section, wherein
regarding a measurement sample which has been prepared by forming, on a capture bead, an immune complex of a phosphorylated tau protein in a biological sample collected from a subject, a detection antibody, and a capture antibody in the presence of a non-capture bead and which contains the non-capture bead and the capture bead to which the immune complex binds, the processing section obtains a signal derived from the immune complex,
the processing section estimates, based on intensity of the signal, an amount of a tau protein accumulated in a brain of the subject which amount is quantified by tau PET,
the processing section obtains information for evaluating a degree of progression of a tauopathy in the subject, based on the amount of the tau protein accumulated in the brain of the subject which amount has been estimated,
the non-capture bead does not bind to the immune complex,
one of the detection antibody and the capture antibody is an antibody which recognizes an amino acid sequence from position 151 to position 244 in an amino acid sequence represented by SEQ ID NO. 1,
the other of the detection antibody and the capture antibody is an antibody which recognizes a phosphorylated epitope including a phosphorylated amino acid residue in the amino acid sequence represented by SEQ ID NO. 1, and
the intensity of the signal does not correlate with an amount of amyloid accumulated in the brain of the subject which amount is quantified by amyloid PET.

16. (canceled)

17. (canceled)

18. (canceled)

19. (canceled)
